(19) United States
(12) Patent Application Publication
(10) Pub. No.: US 2022/0028498 A1
(43) Pub. Date: Jan. 27, 2022

(54) METHODS AND SYSTEMS FOR HIGH-THROUGHPUT PATHOGEN TESTING

(71) Applicant: Laboratory Corporation of America Holdings, Burlington, NC (US)

(72) Inventors: David Wilson, Avon, CT (US); Brian Krueger, Burlington, NC (US); Robert Kays, Raleigh, NC (US); David Craig Garritt, Tolland, CT (US)

(21) Appl. No.: 17/443,124

(22) Filed: Jul. 21, 2021

Related U.S. Application Data
(60) Provisional application No. 63/054,518, filed on Jul. 21, 2020.
(60) Provisional application No. 63/064,191, filed on Aug. 11, 2020.
(60) Provisional application No. 63/092,554, filed on Oct. 16, 2020.

(51) Int. Cl. G16B 50/30; G16B 50/10; G01N 33/487; G01N 33/569; G01N 33/50; G06K 9/62

(52) U.S. Cl. CPC G16B 50/30; G16B 50/10; G01N 33/4875; G06K 9/6264; G01N 33/56983; G01N 33/5091; G01N 33/48792

(57) ABSTRACT

Disclosed are methods and systems for high-throughput testing of pathogens, and in some instances, testing for SARS-CoV-2. For example, disclosed is a method for intelligently selecting samples to perform a pooled testing for a pathogen including the steps of obtaining samples from multiple regions/populations, determining a prevalence of the pathogen in the samples from each region/population, determining an optimal selection plan to perform the pooled testing, selecting and combining samples based on the optimal selection plan, aliquoting the samples in the combined sample set based on the optimal selection plan, pooling and testing the samples in the combined sample set based on the optimal pooling design to determine a presence or absence of a detectable amount of the pathogen in each of the pooled samples, and determining whether at least one individual sample comprises the detectable amount of the pathogen.

### CROSS-REFERENCE TO RELATED APPLICATIONS

The present application claims the benefit and priority of U.S. Provisional Application No. 63/092,554, filed on Oct. 16, 2020, U.S. Provisional Application No. 63/064,191, filed on Aug. 11, 2020, and U.S. Provisional Application No. 63/054,518, filed on Jul. 21, 2020, which are hereby incorporated by reference in their entireties for all purposes

### FIELD

The present disclosure relates to sample pooling, and in particular to techniques for high-throughput testing of pathogens, and in some instances, testing for COVID-19.

### BACKGROUND

The SARS-CoV-2 can cause a serious or life-threatening disease or condition, including severe respiratory illness, to humans infected by this virus. On Feb. 11, 2020, the virus tentatively named 2019-nCoV was formally designated as Severe acute respiratory syndrome coronavirus 2 (SARS-CoV-2). Also on Feb. 11, 2020, the disease caused by SARS-CoV-2 was formally designated as Coronavirus Disease 2019 (COVID-19). On Feb. 4, 2020, the Secretary of the Department of Health and Human Services (HHS) determined that there is a public health emergency that has a significant potential to affect national security or the health and security of United States citizens living abroad, and that involves the virus that causes COVID-19. Thus, there is a need for the development of methods and systems for the detection of COVID-19.

Sample pooling is a method for performing very high throughput testing whereby patient samples are combined together and tested as pools. Sample pooling can be important when demand for testing exceeds capacity and/or when reagent and consumables become limiting. Pooling may also be very useful in populations with low prevalence disease. If a sample pool tests positive, samples are retested to determine which individual within the pool was positive. Pooling, however, does have its limitations in that if done incorrectly, can increase the overall number of tests required for confirmation of a positive result thereby reducing throughput. Thus, there is a need to develop methods and systems for sample pooling.

### SUMMARY

In various embodiments, a method is provided for high-throughput testing for a pathogen. The method comprises: selecting multiple samples to be used in a pooling system for testing the multiple samples for the pathogen using a testing assay, where the multiple samples are obtained from multiple subjects within one or more regions or populations; obtaining a prevalence of the pathogen in the multiple samples; identifying a pooled testing protocol for the pooling system, where the identifying comprises: generating a plurality of potential multidimensional matrices for testing the multiple samples for the pathogen, where each potential multidimensional matrix provides for column, row, and/or address based pooling of the multiple samples, and a size of the potential multidimensional matrix is determined by a number of samples in the columns, rows, and/or addresses that is selected based on a sensitivity of the testing assay for the pathogen; determining for each potential multidimensional matrix a number of initial tests to be performed based on the size of the potential multidimensional matrix; predicting for each potential multidimensional matrix a number of retests to be performed based on a predicted number of positive samples in the potential multidimensional matrix and a predicted arrangement of the positives within the potential multidimensional matrix, where the predicted number of positive samples is determined based on a discrete probability calculated for each possible number of positives based on the prevalence of the pathogen in the population to be tested, and the predicted arrangement of the positives is determined based on a discrete probability calculated for each possible positive arrangement occurring within the potential multidimensional matrix; predicting for each potential multidimensional matrix a total number of tests to be performed based on the number of initial tests and the number of retests; comparing the predicted total number of tests to be performed for each potential multidimensional matrix against the predicted total number of tests to be performed for all other potential multidimensional matrices within the plurality of potential multidimensional matrices; and selecting, based on the comparison, a multidimensional matrix with a least total number of tests to be performed to form a basis for the pooled testing protocol; aliquoting the multiple samples in the multidimensional matrix based on the pooled testing protocol; pooling samples from each column, row, and/or address of the multidimensional matrix; testing the pooled samples with the testing assay to determine a presence or absence of a detectable amount of the pathogen in each of the pooled samples; and determining, based on the presence or absence of the detectable amount of the pathogen in each of the pooled samples, whether at least one individual sample comprises the detectable amount of the pathogen.

In some embodiments, the at least one individual sample that comprises the detectable amount of the pathogen is identified as an unequivocal sample that is common to a row and column or a row, column, and address of pooled samples that each comprises a detectable amount of the pathogen when at least one of followings happens: (i) a number of positive rows is one, (ii) a number of positive columns is one, or (iii) a number of positive address is one.

In some embodiments, the method further comprises retesting individual samples identified as equivocally positive or potentially positive for comprising the detectable amount of the pathogen, where each of the individual samples that comprises the detectable amount of the pathogen is identified as equivocally positive or potentially positive that is common to a row and column or a row, column, and address of pooled samples that each comprises a detectable amount of the pathogen when each number of positive rows, positive columns, and positive address is not one.

In some embodiments, the size of the potential multidimensional matrix is selected to limit a number of positive samples per matrix.

In some embodiments, the size of the potential multidimensional matrix is selected to provide about one positive sample per matrix.

In some embodiments, the multidimensional matrix is a physical array of the samples.

In some embodiments, the multidimensional matrix is an in silico array of the samples.

In some embodiments, the multidimensional matrix is two-dimensional (2D).

In some embodiments, the multidimensional matrix is three-dimensional (3D).

In some embodiments, the pathogen is one of a virus, a bacteria, a fungus, a protozoa or an algae. In some embodiments, the testing comprises detection of a nucleic acid from the pathogen.

In some embodiments, the detection comprises amplification. In some embodiments, the amplification comprises real-time reverse transcription PCR (RT-PCR).

In some embodiments, the pathogen is SARS-CoV-2. In some embodiments, a nucleic acid from a SARS-CoV-2 nucleocapsid (N) gene sequence is detected.

In some embodiments, the multiple samples are biological samples. In some embodiments, the multiple samples comprise a specimen from either an upper or lower respiratory system. In some embodiments, the multiple samples comprise at least one of a nasopharyngeal swab, an oropharyngeal swab, sputum, a lower respiratory tract aspirate, a bronchoalveolar lavage, a nasopharyngeal wash and/or aspirate or a nasal aspirate.

In some embodiments, the multidimensional matrix is a 5 by 5 array of samples.

In some embodiments, the multidimensional matrix is a 4 by 4 array of samples.

In some embodiments, the testing comprises detection of a protein from the pathogen.

In some embodiments, the testing comprises detection of an antibody response to the pathogen.

In some embodiments, selecting the multiple samples to be used in the pooling system is based at least in part on an origin of the sample.

In some embodiments, selecting the multiple samples to be used in the pooling system is based at least in part on an expected disease prevalence.

In some embodiments, the obtaining the prevalence of the pathogen in the multiple samples comprises estimating the prevalence of the pathogen in the multiple samples.

In some embodiments, the sizes of one or more matrices of the plurality of the potential multidimensional matrices are different from those of other matrices of the plurality of the potential multidimensional matrices.

In various embodiments, a method is provided for designing a pooled testing protocol for a pathogen. The method comprises: obtaining a plurality of sets of multiple samples to be used for the pooled testing for the pathogen using a testing assay, where the multiple samples in each set of the plurality of sets are obtained from multiple subjects within a same region or population, and the multiple samples in different sets are obtained from the multiple subjects within different regions or different populations; obtaining a prevalence of the pathogen in each set of the multiple samples; obtaining a prevalence of the pathogen in a combination of a plurality of sets of the multiple samples; and determining an aliquoting technique to perform a pooled test, where the determining comprises: for each set of the multiple samples and the combination of the plurality of sets of the multiple samples: generating a plurality of potential multidimensional matrices for testing the multiple samples for the pathogen, where each potential multidimensional matrix provides for column, row, and/or address based pooling of the multiple samples, and a size of the potential multidimensional matrix is determined by a number of samples in the columns, rows, and/or addresses that is selected based on a sensitivity of the testing assay for the pathogen; determining for each potential multidimensional matrix a number of initial tests to be performed based on the size of the potential multidimensional matrix; predicting for each potential multidimensional matrix a number of retests to be performed based on a predicted number of positive samples in the potential multidimensional matrix and a predicted arrangement of the positives within the potential multidimensional matrix, where the predicted number of positive samples is determined based on a discrete probability calculated for each possible number of positives based on the prevalence of the pathogen, and the predicted arrangement of the positives is determined based on a discrete probability calculated for each possible positive arrangement occurring within the potential multidimensional matrix; predicting for each potential multidimensional matrix a total number of tests to be performed based on the number of initial tests and the number of retests; comparing the predicted total number of tests to be performed for each potential multidimensional matrix against the predicted total number of tests to be performed for all other potential multidimensional matrices within the plurality of potential multidimensional matrices; and selecting, based on the comparison, a multidimensional matrix with a least total number of tests to be performed to form a basis for the pooled testing protocol; comparing a sum of the least total numbers of tests to be performed for all sets of the multiple samples against a sum of the least total number of tests to be performed for the combination of the plurality of sets of the multiple samples and the least total numbers of tests to be performed for the sets of the multiple samples not in the combination of the plurality of sets of the multiple samples; and selecting, based on the comparison, the multidimensional matrices with the least sum to form a basis for the pooled testing protocol.

In some embodiments, the obtaining the prevalence of the pathogen in the combination of the plurality of sets of the multiple samples comprises estimating the prevalence of the pathogen in the combination of the plurality of sets of the multiple samples based on the prevalence of the pathogen in each set of the plurality of sets of the multiple samples.

In some embodiments, the sizes of one or more matrices of the plurality of the potential multidimensional matrices are different from those of other matrices of the plurality of the potential multidimensional matrices.

In some embodiments, the size of the potential multidimensional matrix is selected to limit a number of positive samples per matrix.

In various embodiments, a method is provided for intelligently selecting samples to perform a pooled testing for a pathogen. The method comprises: obtaining samples from a plurality of regions or populations, where the samples from each region or population form a sample selection candidate set; determining a prevalence of the pathogen in the samples from each region or population of the plurality of regions or populations; determining, by an intelligent selection machine, an optimal selection plan to perform the pooled testing on the samples, where the optimal selection plan comprises an optimal ratio to combine the samples from the plurality of regions or populations, an optimal prevalence in a combined sample set, and an optimal pooling design for the pooled testing; selecting samples from one or more sample selection candidate set based on the optimal ratio; combining the selected samples to form the combined sample set with the optimal prevalence; aliquoting the samples in the combined sample set based on the optimal pooling design; pooling the samples in the combined sample set based on the optimal pooling design; testing the pooled samples to determine a presence or absence of a detectable amount of the pathogen in each of the pooled samples; and determining, based on the presence or absence of the detectable amount of the pathogen in each of the pooled samples, whether at least one individual sample comprises the detectable amount of the pathogen.

In some embodiments, the intelligent selection machine is configured to perform: obtaining sample set information, where the sample set information comprises a size of each sample set and a prevalence of a pathogen in each sample set; obtaining a pooled testing objective function; determining a set of possible pooling sizes and a set of possible prevalence of the pathogen based on the sample set information; determining a number of initial tests to be performed for a possible pooling size in the set of the possible pooling sizes; predicting a number of retests to be performed for a combination of a possible pooling size in the set of the possible pooling sizes and a possible prevalence in the set of the possible prevalence; and determining an optimal selection plan based on the pooled testing objective function, where the optimal selection plan comprises an optimal ratio to combine samples in one or more sample sets, an optimal prevalence in a combined sample set, and an optimal pooling design for the pooled testing.

In some embodiments, the set of the possible pooling sizes is determined based on (i) a sensitivity of a testing assay, (ii) a specification of a testing assay, (iii) the prevalence of the pathogen, (iv) a policy requirement, or (v) any combination thereof.

In some embodiments, the set of the possible prevalence of the pathogen is determined based on the prevalence of the pathogen in each sample set, where a maximum possible prevalence is less than or equal to a largest prevalence of the pathogen in all sample sets, and a minimum possible prevalence is greater than or equal to a smallest prevalence of the pathogen in all sample sets.

In some embodiments, the pooled testing objective function is (i) a function to minimize a number of total tests, (ii) a function to minimize a number of retests, or (iii) a function to minimize a total cost.

In some embodiments, the determining the number of the initial tests to be performed comprises calculating a number of pools corresponding to the possible pooling size.

In some embodiments, the predicting the number of the retests to be performed for the combination of the possible pooling size in the set of the possible pooling sizes and the possible prevalence in the set of the possible prevalence comprises calculating an expected number of retests based on the possible prevalence for the possible pooling size according to a pooling design and providing the expected number of the retests.

In some embodiments, the pooling design is a matrix pooling, a double pooling, a triple pooling, and/or a non-square pooling.

In some embodiments, the determining the optimal selection plan comprises: determining a value of the pooled testing objective function for a combination of a possible pooling size and a prevalence; determining an optimal combination of an optimal pooling size and an optimal prevalence, where the optimal combination of the optimal pooling size and the optimal prevalence yields a greatest or a smallest value of the pooled testing objective function; determining an optimal ratio to combine samples in one or more sample sets to form a combined sample set, where a prevalence in the combined sample set equals to the optimal prevalence; determining an optimal pooling design for the pooled testing, where the optimal pooling design comprises the optimal pooling size; and providing an optimal selection plan, where the optimal selection plan comprises the optimal ratio to combine the samples in the one or more sample sets, the optimal prevalence in the combined sample set, and the optimal pooling design for the pooled testing.

In some embodiments, the samples comprise a specimen from either an upper or lower respiratory system.

In some embodiments, the samples comprise at least one of a nasopharyngeal swab, an oropharyngeal swab, sputum, a lower respiratory tract aspirate, a bronchoalveolar lavage, a nasopharyngeal wash and/or aspirate or a nasal aspirate.

In some embodiments, the obtaining the samples comprises collecting the samples from a plurality of collection sites.

In some embodiments, the pathogen is SARS-CoV-2.

In some embodiments, the determining the prevalence of the pathogen in the samples from each region or population of the plurality of regions or populations comprises estimating the prevalence from a historical record in each region or population.

In some embodiments, the pooled testing comprises a matrix pooling, a double pooling, a triple pooling, and/or a non-square pooling.

In some embodiments, the double pooling comprises: determining a number of pools to be performed in the pooled testing; and pooling samples and testing the samples in each pools, where each pair of the pools overlaps in at most a predetermined number of samples, and where each sample is in exactly two pools.

In some embodiments, the triple pooling comprises: determining a number of pools to be performed in the pooled testing; and pooling samples and testing the samples in each pools, where each pair of the pools overlaps in at most a predetermined number of samples, and where each sample is in exactly three pools.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows an example of a 2D matrix pooling technique that includes sixteen samples arranged in a 4×4 matrix in accordance with various embodiments of the disclosure.

FIG. 2 shows initial tests required for various testing protocols in accordance with various embodiments of the disclosure.

FIG. 3A shows an example of a 1D Pooling (1×5) protocol having equivocal samples in accordance with various embodiments of the disclosure.

FIG. 3B shows an example of a 2D Pooling (4×4) protocol having unequivocal samples in accordance with various embodiments of the disclosure.

FIG. 3C shows an example of a 1D Pooling (4×4) protocol having equivocal samples in accordance with various embodiments of the disclosure.

FIG. 4A illustrates the likelihood of a pool being positive and consequently the number of retests to be performed can be predicted for 1D pooling using a binomial distribution in accordance with various embodiments of the disclosure.

FIG. 4B shows the total tests provided on the y-axis that can be predicted for resolving 1000 samples dependent upon the pathogen prevalence provided on the x-axis in accordance with various embodiments of the disclosure.

FIGS. 5A-5C illustrate that the number of positive samples in a 2D matrix is determinable from a binomial distribution for a given prevalence and the arrangement of positives samples within the 2D matrix is determinable from a probability tree in accordance with various embodiments of the disclosure.

FIG. 6 illustrates how a binomial distribution is calculated for the entire prevalence range to be analyzed in accordance with various embodiments of the disclosure.

FIG. 7 illustrates how the arrangement of positives within the matrix is determinable from a probability tree in accordance with various embodiments of the disclosure.

FIGS. 8A-8C illustrate for a given number of positives, there are n matrix arrangements, and the average number of retests required may be calculated in accordance with various embodiments of the disclosure.

FIG. 9 shows a comparison of the total number of tests (initial tests and retests) to result in 1,000 samples for a given prevalence in accordance with various embodiments of the disclosure. 2D—4×4 pooling corresponds to two dimensional pooling as a 4×4 matrix; and 2D—5×5 pooling corresponds to two dimensional pooling as a 5×5 matrix as disclosed herein.

FIG. 10 shows a system in accordance with an embodiment of the disclosure used to perform a sample pooling method in accordance with various embodiments of the disclosure.

FIG. 11 shows a comparison of expected total tests of 1000 samples for different pooling methods for a given prevalence in accordance with various embodiments of the disclosure. 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 12 shows a comparison of expected retests of 1,000 samples for different pooling methods for a given prevalence in accordance with various embodiments of the disclosure. 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 13 shows a comparison of the percentage (Pct) (%) of total tests that are retests for different pooling methods for a given prevalence in accordance with various embodiments of the disclosure. The percentage of Tests that are Retests=(#retests)/(#initial tests+#retests). 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 14 shows a comparison of the percentage of samples that are unequivocally resulted (identified as positive or negative) on the 1^{st} test for different pooling methods for a given prevalence in accordance with various embodiments of the disclosure. The percentage of Samples Determined on 1^{st} Test=(#initial test)/(#initial tests+#retests). 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 15 shows a cost factor analysis to retests where the factor for retests is 1.5 in accordance with various embodiments of the disclosure for individual samples vs pooled samples. 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 16 shows a cost factor analysis to retests where the factor for retests is 2.0 in accordance with various embodiments of the disclosure for individual samples vs pooled samples. 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 17 shows a cost factor analysis to retests where the factor for retests is 3.0 in accordance with various embodiments of the disclosure for individual samples vs pooled samples. 1D—1×5 pooling corresponds to the pooling of five individual samples; 2D—4×4 pooling corresponds to two dimensional pooling as a 4×4 matrix; 2D—5×5 pooling corresponds to two dimensional pooling as a 5×5 matrix as disclosed herein.

FIG. 18 shows an average total test time analysis where the factor for retesting is 1.0 in accordance with various embodiments of the disclosure. 1D—1×5 1 pooling corresponds to the pooling of five individual samples; 2D—4×4 1 pooling corresponds to two dimensional pooling as a 4×4 matrix; 2D—5×5 1 pooling corresponds to two dimensional pooling as a 5×5 matrix as disclosed herein.

FIG. 19 shows an average total test time analysis where the factor for retesting is 1.5 in accordance with various embodiments of the disclosure. 1D—1×5 1.5 pooling corresponds to the pooling of five individual samples; 2D—4×4 1.5 pooling corresponds to two dimensional pooling as a 4×4 matrix; 2D—5×5 1.5 pooling corresponds to two dimensional pooling as a 5×5 matrix as disclosed herein.

FIG. 20 shows an average total test time analysis where the factor for retesting is 2.0 in accordance with various embodiments of the disclosure. 1D—1×5 2 pooling corresponds to the pooling of five individual samples; 2D—4×4 2 pooling corresponds to two dimensional pooling as a 4×4 matrix; 2D—5×5 2 pooling corresponds to two dimensional pooling as a 5×5 matrix as disclosed herein.

FIG. 21 shows a combination of two 96 well plates with 92 real-time samples on each plate in accordance with various embodiments. The combined sample set may further be pooled in two pool sets.

FIG. 22 shows a combination of two 96 well plates with 88 real-time samples on each plate in accordance with various embodiments. The combined sample set may further be pooled in each row.

FIG. 23 shows a double pooling design with 10 pools of size 4 using a graph with 10 vertices and 20 edges in accordance with various embodiments.

FIG. 24 shows one instance where a double pooling design with 10 pools of size 4 yields both unequivocally positive results and equivocally positive results in accordance with various embodiments.

FIG. 25 shows using a subgraph construction method to provide a number of retests under a double pooling design in accordance with various embodiments.

FIG. 26 shows a comparison of total tests numbers among different pooling techniques in accordance with various embodiments. 4×4 (Matrix) pooling corresponds to two dimensional pooling as a 4×4 matrix; 5×5 (Matrix) pooling corresponds to two dimensional pooling as a 5×5 matrix; 4×4 (Double Pooling) pooling corresponds to a double pooling with 4 samples in each pool; 5×5 (Double Pooling) pooling corresponds to a double pooling with 5 samples in each pool as disclosed herein.

FIG. 27 is a flowchart illustrating a process for performing intelligent sample selection and pooled testing in accordance with various embodiments.

FIG. 28 is a flowchart illustrating a process for performing functions configured in an intelligent selection machine in accordance with various embodiments.

FIG. 29 illustrates one exemplary embodiment to a method using a decision graph to determine an optimal selection plan in accordance with various embodiments. 1D—1×5 pooling corresponds to the pooling of five individual samples; 4×4 (Matrix) pooling corresponds to two dimensional pooling as a 4×4 matrix; 5×5 (Matrix) pooling corresponds to two dimensional pooling as a 5×5 matrix; 4×4 (Double Pooling) pooling corresponds to a double pooling with 4 samples in each pool; 5×5 (Double Pooling) pooling corresponds to a double pooling with 5 samples in each pool as disclosed herein.

FIG. 30 shows the average Ct difference in accordance with various embodiments of the disclosure. The average Ct difference from the original N1/N2 Ct and the pooled N1/N2 Ct was calculated for both N=4 and N=5 pools. Error bars are standard deviation.

FIG. 31 shows histograms of N1 and N2 Cts for 148,550 clinical samples in accordance with various embodiments of the disclosure. N1—Blue (Left panel), N2—Red (Right panel).

FIG. 32 shows N=4 Passing-Bablock Analysis in accordance with various embodiments of the disclosure.

FIG. 33 shows N=5 Passing-Bablock Analysis in accordance with various embodiments of the disclosure.

FIG. 34 shows a 4×4 matrix in accordance with various embodiments of the disclosure, where Arrows indicate pooling direction. Boxes outside matrix grid represent the final pools.

FIG. 35 shows an unequivocal positive sample identification in a 4×4 matrix in accordance with various embodiments of the disclosure.

FIG. 36 shows an unequivocal identification in a 4×4 matrix in accordance with various embodiments of the disclosure, when 2 samples are positive, where red (darker shading) indicates a positive sample or pool.

FIG. 37 shows an equivocal identification in a 4×4 matrix in accordance with various embodiments of the disclosure, when 2 samples are positive, where red (darker shading) indicates a positive sample or pool.

FIG. 38 shows an equivocal identification in a 4×4 matrix in accordance with various embodiments of the disclosure, when no samples are positive. This can occur when 1 or 2 pools are positive without a corresponding row or column resulting positive. Red (darker shading) indicates a positive sample or pool.

FIG. 39 shows a system for high-throughput pooling in accordance with various embodiments of the disclosure used to perform a method in accordance with an embodiment of the disclosure.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

Sample pooling and subsequent pooled testing is a procedure where individual specimens (e.g., urine or blood) are combined into a pooled specimen to test for a response (e.g., a binary response such as positive or negative status). In the most widely used form of pooled testing known as “Dorfman testing,” pools that test negative have all individuals within them declared negative. Pools that test positive indicate that at least one individual within each pool is positive, and individual retesting of each specimen is subsequently used to decode the positives from the negatives. The strong appeal of pooled testing is that it can significantly reduce the number of tests and associated costs when the prevalence for a disease is small. This has led to the application of pooled testing in a wide variety of infectious disease screening settings, such as blood donation screening by the American Red Cross, chlamydia and gonorrhea opportunistic testing in medical clinics, influenza surveillance through blood donations, and West Nile virus surveillance in mosquitoes.

While Dorfman testing is the easiest to apply, it usually leads to the largest number of tests needed among all pooled testing procedures. Rather than testing all members of a positive pool individually, there are a number of alternative techniques that have been developed to minimize the total number of tests performed within the pooling. For example, in the halving technique a positive pool can be split into two or more sub-pools. If any sub-pool tests positive, further splitting or individual testing can be performed on it. Another alternative to immediate individual testing for a positive pool is the Sterrett's technique, which includes exploiting the fact that there is most likely a very small number of positives within properly sized pools (often, there is only one positive per pool). For an initial pool that tests positive, individuals may be retested at random one-by-one until the first positive individual is found. Once the first positive is found, individuals that have not been retested are re-pooled and tested again. Retesting ends if this new pool tests negative. Lastly, matrix or array testing, is a pooled testing procedure often used with high throughput screening. Unlike halving and Sterrett's procedures, where individuals are assigned to one initial pool, individuals are assigned to two separate pools. This is done by constructing a matrix-like grid of specimens and pooling individuals within rows and within columns. Specimens lying at the intersections of positive rows and positive columns are tested individually to decode the positives from the negatives.

However, non-informative techniques (meaning it does not account for extra information available within a heterogeneous population) as described above typically lead to the largest number of tests needed among all pooled testing procedures. In order to further minimize the total number of tests performed, a number of informative techniques (meaning it does account for the extra information available within a heterogeneous population) have been developed. Informative procedures rely on the basic idea that individuals have different risks of being positive. These risks can be measured in a number of ways and applied to the current individuals being screened in order to estimate their risk probability of having a disease. These probabilities may then be used to select pool sizes, set up testing to minimize the number of positive pools, and/or determine the order in which individuals are retested within a positive pool. With respect to accuracy, pooled testing using non-informative and informative techniques typically improves upon the overall pooling specificity and pooling positive predictive value when compared to individual testing. However, pooling sensitivity and pooling negative predictive values can be much lower when the assay sensitivity is low. Moreover, there is not one pooled testing technique that is best (in terms of number of tests and accuracy) all of the time. Prevalence levels, assay accuracy, availability of risk factor information, and risk probability distributions all play roles in determining which technique will work best for a given assay.

To address these limitations and problems, the pooled sampling and testing techniques described herein improve upon accuracy and decrease testing time per sample. This can be important with assays requiring a large volume of samples such as current COVID-19 assays, by improving upon pooled matrix or array techniques using deterministic factors including number of positive sample predicted within a matrix and predicted arrangement of positives within the matrix. In various embodiments, a method is provided for high-throughput testing for a pathogen. The method includes selecting multiple samples to be used in a pooling system for testing the multiple samples for the pathogen using a testing assay. The multiple samples are obtained from multiple subjects within one or more geographic regions or populations. The method further comprises obtaining a prevalence of the pathogen in the multiple samples, and identifying a pooled testing protocol for the pooling system.

In certain instances, the identifying comprises: generating a plurality of potential multidimensional matrices for testing the multiple samples for the pathogen, where each potential multidimensional matrix provides for column, row, and/or address based pooling of the multiple samples, and a size of the potential multidimensional matrix is determined by a number of samples in the columns, rows, and/or addresses that is selected based on a sensitivity of the testing assay for the pathogen; determining for each potential multidimensional matrix a number of initial tests to be performed based on the size of the potential multidimensional matrix; predicting for each potential multidimensional matrix a number of retests to be performed based on a predicted number of positive samples in the potential multidimensional matrix and a predicted arrangement of the positives within the potential multidimensional matrix, where the predicted number of positive samples is determined based on a discrete probability calculated for each possible number of positives based on the prevalence of the pathogen in the population to be tested, and the predicted arrangement of the positives is determined based on a discrete probability calculated for each possible positive arrangement occurring within the potential multidimensional matrix; predicting for each potential multidimensional matrix a total number of tests to be performed based on the number of initial tests and the number of retests; comparing the predicted total number of tests to be performed for each potential multidimensional matrix against the predicted total number of tests to be performed for all other potential multidimensional matrices within the plurality of potential multidimensional matrices; and selecting, based on the comparison, a multidimensional matrix with a least total number of tests to be performed to form a basis for the pooled testing protocol.

In some embodiments, the method further includes aliquoting the multiple samples in the multidimensional matrix based on the pooled testing protocol; pooling samples from each column, row, and/or address of the multidimensional matrix; testing the pooled samples with the testing assay to determine a presence or absence of a detectable amount of the pathogen in each of the pooled samples; and determining, based on the presence or absence of the detectable amount of the pathogen in each of the pooled samples, whether at least one individual sample comprises the detectable amount of the pathogen.

In other embodiments, a method is provided for designing a pooled testing protocol for a pathogen. The method comprises obtaining a plurality of sets of multiple samples to be used for the pooled testing for the pathogen using a testing assay. The multiple samples in each set of the plurality of sets are obtained from multiple subjects within a same region or population, and the multiple samples in different sets are obtained from the multiple subjects within different regions or different populations. The method further comprises obtaining a prevalence of the pathogen in each set of the multiple samples; obtaining a prevalence of the pathogen in a combination of a plurality of sets of the multiple samples; and determining an aliquoting technique to perform a pooled test.

The determining the aliquoting technique comprises: for each set of the multiple samples and the combination of the plurality of sets of the multiple samples: generating a plurality of potential multidimensional matrices for testing the multiple samples for the pathogen, where each potential multidimensional matrix provides for column, row, and/or address based pooling of the multiple samples, and a size of the potential multidimensional matrix is determined by a number of samples in the columns, rows, and/or addresses that is selected based on a sensitivity of the testing assay for the pathogen; determining for each potential multidimensional matrix a number of initial tests to be performed based on the size of the potential multidimensional matrix; predicting for each potential multidimensional matrix a number of retests to be performed based on a predicted number of positive samples in the potential multidimensional matrix and a predicted arrangement of the positives within the potential multidimensional matrix, where the predicted number of positive samples is determined based on a discrete probability calculated for each possible number of positives based on the prevalence of the pathogen, and the predicted arrangement of the positives is determined based on a discrete probability calculated for each possible positive arrangement occurring within the potential multidimensional matrix; predicting for each potential multidimensional matrix a total number of tests to be performed based on the number of initial tests and the number of retests; comparing the predicted total number of tests to be performed for each potential multidimensional matrix against the predicted total number of tests to be performed for all other potential multidimensional matrices within the plurality of potential multidimensional matrices; and selecting, based on the comparison, a multidimensional matrix with a least total number of tests to be performed to form a basis for the pooled testing protocol.

The determining the aliquoting technique further comprises: comparing a sum of the least total numbers of tests to be performed for all sets of the multiple samples against a sum of the least total number of tests to be performed for the combination of the plurality of sets of the multiple samples and the least total numbers of tests to be performed for the sets of the multiple samples not in the combination of the plurality of sets of the multiple samples; and selecting, based on the comparison, the multidimensional matrices with the least sum to form a basis for the pooled testing protocol.

In yet other embodiments, a method is provided for performing a matrix pooled testing for a pathogen. The method comprises: obtaining multiple samples to be used in the matrix pooled testing for the pathogen using a testing assay, where the multiple samples are obtained from multiple subjects within one or more regions or populations; obtaining a size of a matrix to be used in the matrix pooled testing for the pathogen, where the size of the matrix is determined by a pooled testing protocol for the pathogen; aliquoting the multiple samples in the matrix; pooling samples from each column, row, and/or address of the matrix; testing the pooled samples with the testing assay to determine a presence or absence of a detectable amount of the pathogen in each of row pools, column pools, and/or address pools; determining, based on the presence or absence of the detectable amount of the pathogen in each of the row pools, the column pools, and/or the address pools, whether each individual sample at an intersection of positive row pools, column pools, and/or address pools is unequivocally positive; retesting (i) each individual sample at the intersection of the positive row pools, column pools, and/or address pools that is not unequivocally positive to determine a presence or absence of a detectable amount of the pathogen in the individual sample, and/or (ii) each individual sample in each positive pool that has no intersection with all other positive pools; and outputting pathogen detection results based on the determining and the retesting.

It will be appreciated that the pooled sampling and testing techniques disclosed herein are applicable to COVID-19, but the methodologies and techniques are applicable to a wide variety of pathogens, pool sizes, and matrix structures.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

### II. POOLING TECHNIQUES AND INTELLIGENT SAMPLE SELECTION METHOD

II.A. Informed Pooled Matrix and Array Assay Techniques for Minimizing Retesting

In various embodiments, disclosed is a method for high-throughput testing for a pathogen comprising: aliquoting a plurality of samples in a multidimensional matrix; pooling samples from each row and column of the matrix; testing the pooled samples to determine the presence or absence of a detectable amount of the pathogen in each of the pooled samples; and determining, based on the detection of the pathogen in a plurality of the pooled samples, whether at least one individual sample comprises a detectable amount of the pathogen. In an embodiment, the at least one individual sample that comprises a detectable amount of the pathogen is identified as a sample that is common to a row and column of pooled samples that each comprise a detectable amount of the pathogen.

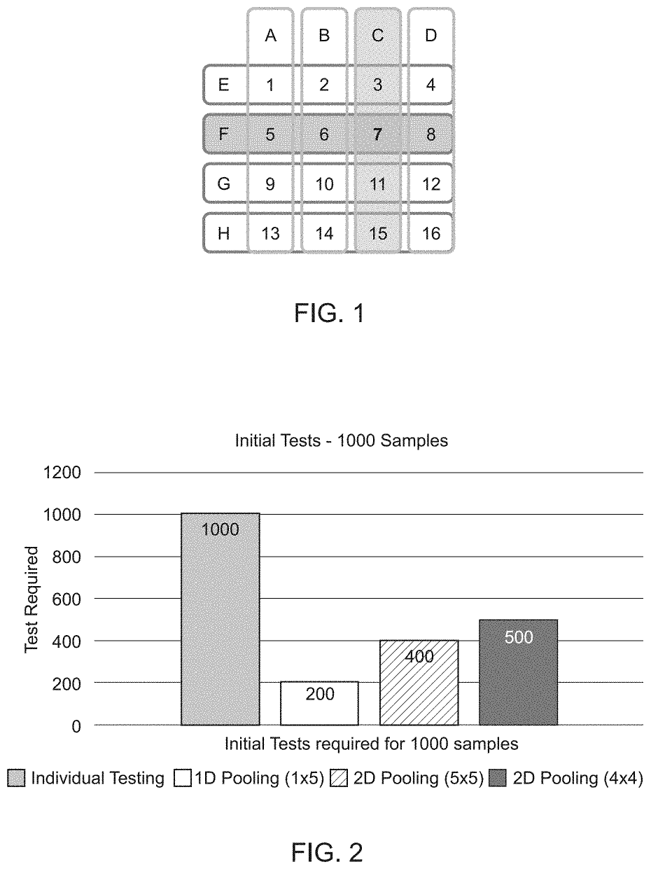

The matrix is simply a determination of how samples are pooled. The matrix may be two-dimensional (2D) or three dimensional (3D) or multi-dimensional. A variety of matrix sizes or arrangements may be used. In an embodiment, the matrix size relates to attributes of the method used for detecting the pathogen including, but not limited to, detection limits, specificity and/or sensitivity. Matrix size may also relate to the volume and sample type. With 2D matrix pooling, samples are arranged in a grid comprising rows and columns. The samples in each row and each column are combined, or pooled. Each sample is a member of exactly two pools. Each pool is then tested. For any pools that test positive, the sample at the intersection of the two pools is marked as either unequivocally positive or equivocally positive. An unequivocally positive sample means the sample is a positive sample, and an equivocally positive sample means the sample has a possibility of being a positive sample. An unequivocally positive sample need not be retested, while each equivocally positive sample needs to be retested. FIG. 1 shows an example of a 2D matrix pooling technique that includes sixteen samples arranged in a 4×4 matrix. Four column pools are created (A-D) and four row pools (E-H). Pools C and F are illustrated as being found to be positive, and consequently sample 7 is determined as positive.

In some embodiments, larger matrices may be used that increase throughput provided that the sensitivity of the method(s) used for detecting the pathogen remains satisfactory and the expected number of positive tests remains below a threshold that would result in the overall number of samples tested being greater based on pooling the samples than it would be testing samples without pooling. For example, the matrix may be a 5 by 5 (5×5) array of samples. Alternatively, the matrix may be a 4 by 4 (4×$) array of samples. In certain embodiments, samples are run twice (i.e., at two different addresses in the matrix) to reduce the need for retesting. Such a matrix may be used for testing for COVID-19 which can be relatively prevalent (>3-5% positivity) in the general population. For example, based on a binomial distribution, at a prevalence of 5% and a pool size of 5, 23% of pools will have a positive sample and with a pool size of 4, 19% of pools will be positive. However, if samples are processed as a 4×4 or 5×5 matrix where each sample is run twice but with different pool members (e.g., with a different arrangement of the samples in a matrix), it is possible in certain instances to ascertain the positive samples without performing retesting of individual members of a positive pool.

In some embodiments, the matrix is a physical array of the samples. For example, for a 2D matrix, samples may be aliquot into wells in a microtiter plate and then samples in each row and column pooled. For a 3D matrix, a plurality of 2D matrices may be assayed in a third dimension. For example, for a 3D matrix, a plurality of microtiter plates (e.g., A1, A2, etc.) may be assayed, such that rows and columns of each plate are assayed in a third pooling that includes samples that have the same address (e.g., assaying all the A1 samples for 5 separate plates, assaying all the A2 samples for 5 separate plates, etc.) in each of the microtiter plates. Alternatively, the matrix need not be physical, but can be an in silico array of the samples whereby a matrix is defined by selecting defined samples for rows and columns and a third dimension based on sample numbering and assignment to a virtual matrix.

The disclosed methods and systems may be used for testing any pathogen. As noted, the methods and systems may be used for detection of a variety of pathogens. In an embodiment, the pathogen is the SARS-CoV-2. In such embodiments, nucleic acid from the SARS-CoV-2 nucleocapsid (N) gene sequence may be detected. In alternate embodiments, the pathogen is one of a virus, a bacteria, a fungus, a protozoa or an algae. The disclosed methods and systems may also be used for detection of various markers and/or biomolecules that are associated with the pathogen of interest. Thus, in certain embodiments, the testing comprises detection of a nucleic acid from the pathogen. Or, the testing may comprise detection of a protein from the pathogen. The testing may also comprise detection of an antibody response to the pathogen. Or the disclosed methods and systems may be applied to detection of other types of biomarkers. For example, for nucleic acid detection, amplification such as PCR may be used. In certain embodiments, the amplification comprises real-time reverse transcription PCR (RT-PCR), the products of which may be the subject of detection.

The methods and systems of the disclosure may be applied to a variety of sample types. In certain embodiments, the sample comprises a biological sample. In some embodiments, the biological sample is taken from a subject. In some embodiments, the subject may be a human subject. In some embodiments of the method, the subject may be suspected to have been exposed to any pathogen of interest. In certain embodiments, the pathogen is SARS-CoV-2. As used herein, the terms “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse or sheep) and a primate (e.g., a monkey, such as a cynomolgus monkey, gorilla, chimpanzee or a human).

“Sample” or “patient sample” or “biological sample” or “specimen” are used interchangeably herein. The source of the sample may be solid tissue as from a fresh tissue, frozen and/or preserved organ or tissue or biopsy or aspirate. The source of the sample may be a liquid sample. Non-limiting examples of liquid samples include cell-free nucleic acid, blood or a blood product (e.g., serum, plasma, or the like), urine, nasal swabs, biopsy sample (e.g., liquid biopsy for the detection of cancer or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue sampler other liquid aspirate. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis. For example, in certain embodiments (e.g., COVID-19) the biological sample comprises a specimen from either the upper or lower respiratory system. In an embodiment, the sample may comprise e.g., at least one of a nasopharyngeal swab, a mid-turbinate swab, anterior nares swab, an oropharyngeal swab, sputum, a lower respiratory tract aspirate, a bronchoalveolar lavage, a nasopharyngeal wash and/or aspirate or a nasal aspirate.

Thus, disclosed are systems and methods for high-throughput testing for pathogens. In an embodiment, the systems and methods comprise pooling of samples. In some embodiments, the pools are processed as two dimensional (2D) matrices to eliminate retesting when testing population prevalence is low. Additionally and/or alternatively, the pools are processed as 3D matrices to eliminate retesting when testing population prevalence is low and the sensitivity of the assay allows for detection upon sample dilution (i.e., pooling). In this way, a positive sample will be associated with a single address. By matching the 2D or 3D coordinate with the positive sample, the number of samples that need to be retested is reduced. For example, if 5 samples are pooled, and a positive result is obtained, all 5 samples will need to be retested. In contrast, if samples are arranged in a 2D array (e.g., 5×5) then a positive sample from a particular row can be identified based on which column associated with that row also contains a positive sample.

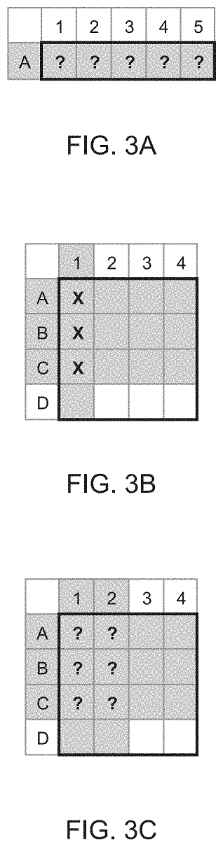

As illustrated in FIG. 2, in general pooling requires fewer initial tests than individual testing. However, in some instances pooling requires retesting due to equivocal results (open to more than one interpretation; ambiguous). In 1D pooling, every sample in a positive pool is equivocal and must be retested. Thus, the number of retests depends on the number of positive pools multiplied by the pool size (i.e., #retests=#positive pools×pool size). For example, as shown in FIG. 3A in a 1D pooling, if a pool A is positive, then all five samples A1-A5 within pool A are equivocal and all five samples (A1-A5) are required to be retested. Whereas in 2D pooling, whether or not a sample in a pool is equivocal and the number of retests required depends on the number of samples in the matrix and the arrangement of positives within the matrix. For example, as shown in FIG. 3B in a 2D pooling with a 4×4 matrix, if pools A, B, C, and 1 are positive but pools D, 2, 3 and 4 are not positive, then samples A1, B1, and C1 are unequivocal and no retests are required. However, as shown in FIG. 3C in a same 2D pooling with a 4×4 matrix, if pools A, B, C, 1, and 2 are positive (and pools D, 3 and 4 are not positive), then samples A1, B1, C1, A2, B2, and C2 are equivocal and six retests are required to definitively identify positive samples.

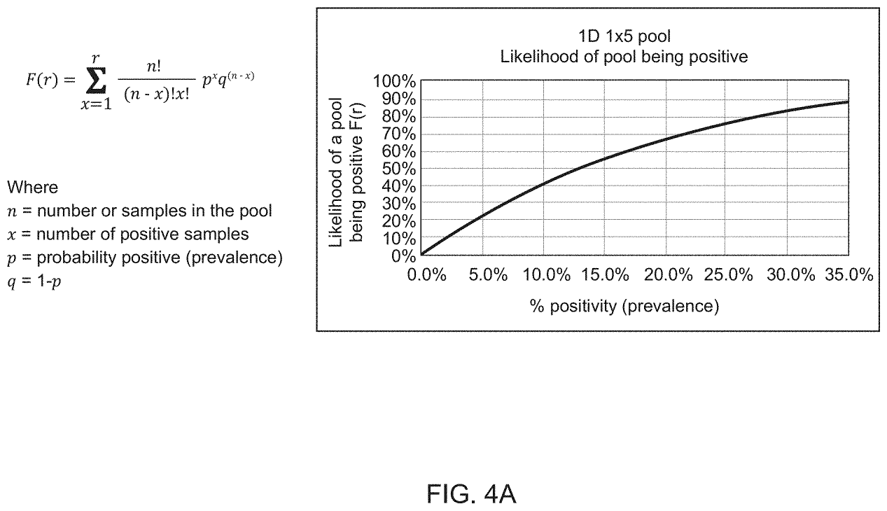

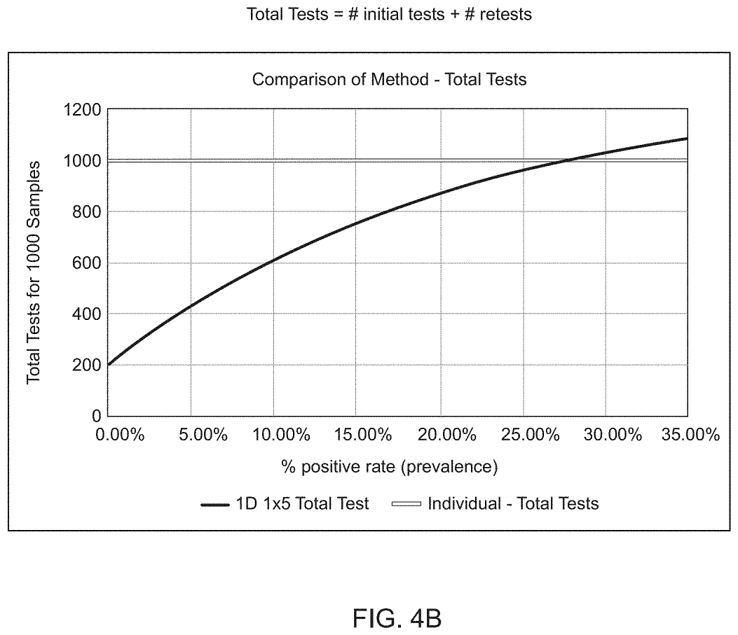

As discussed above, the likelihood of retests in 1D pooling depend on the pathogen prevalence (% positivity rate) and the pool size. The number of retests in 1D pooling is the number of positive pools multiplied by the pool size. The number of positive pools depends on the pathogen prevalence (% positivity rate), and the likelihood of a pool being positive and consequently the number of retests to be performed can be predicted using a binomial distribution, as shown in FIG. 4A. Accordingly, the predicted number of total test (#initial tests+#retests) can be calculated. FIG. 4B shows the total tests provided on the y-axis that can be predicted for resolving 1000 samples dependent upon the pathogen prevalence provided on the x-axis. As shown, for a pathogen prevalence of less than about 27%, a 1D pooling with 1×5 pool requires fewer total tests than individual testing. However, for a pathogen prevalence of greater the about 27%, a 1D pooling with 1×5 pool requires more total tests than individual testing. Thus, a determination can be made on whether to utilize pooled testing versus individual testing based on the predicted number of total test (#initial tests+#retests).

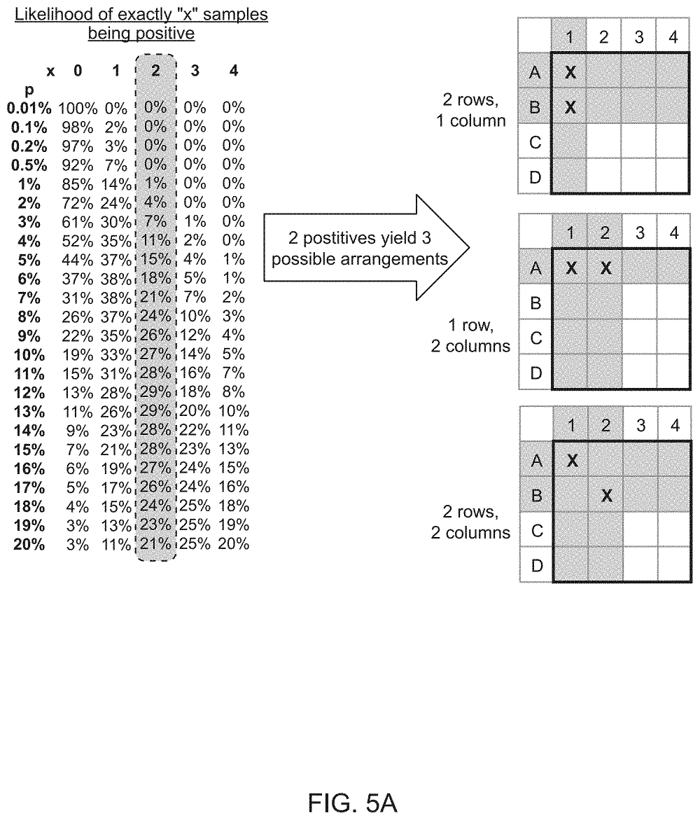

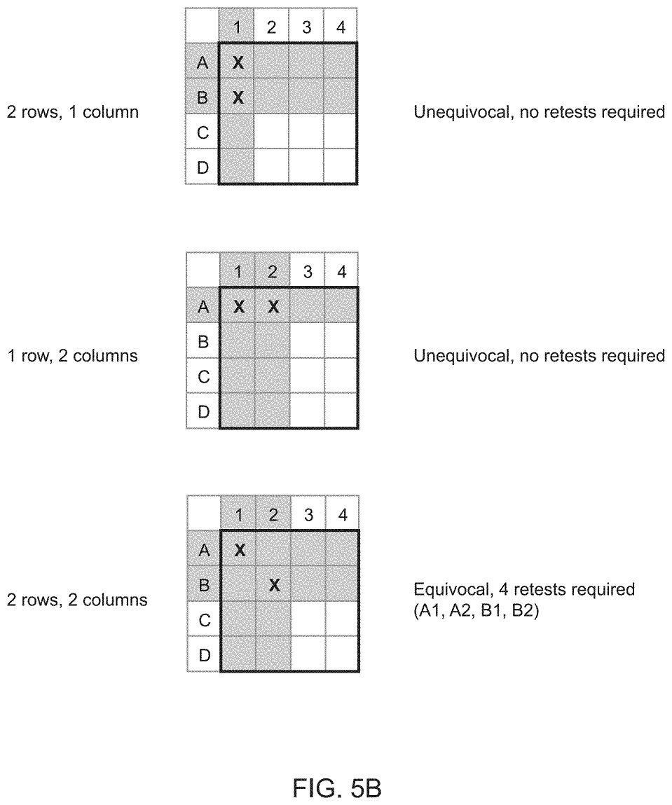

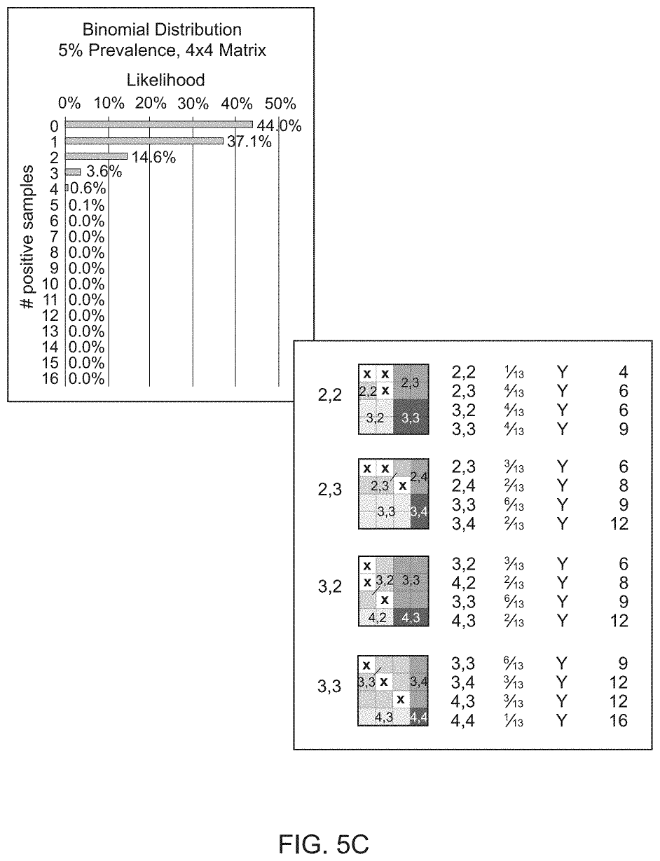

In contrast to 1D pooling, the likelihood of retests in a 2D pooling depends on the number of positive samples in the matrix (determinable from a binomial distribution) and the arrangement of positives within the matrix (determinable from probability tree), as shown in FIGS. 5A and 5B. For example, FIG. 5A illustrates 3 possible arrangements of 2 positive samples within a 4×4 matrix, and FIG. 5B further illustrates whether each arrangement yields equivocal or unequivocal results and whether retests are required. When the 2 positive samples are in two rows of the same column, the initial tests will show the two row pools and the one column pools as positive, and the two samples at the intersections of the two rows and one column are unequivocally positive thus no retests are required, as shown in the top graph of FIG. 5B. Similarly, when the 2 positive samples are in two columns of the same row, the yield result is also unequivocal and no retests are necessary, as shown in the middle graph of FIG. 5B. However, when the 2 positive samples are in two columns of two different rows, as shown in the bottom graph of FIG. 5B, the four samples (A1, A2, B1, and B2) at intersections of the two rows and two columns are equivocal, because any sample could be positive (e.g., the initial test result may be yielded by A1 and B2 positive, or A2 and B1 positive, or any three of the four samples positive, or all four sample positive). Therefore, 4 retests are required under this circumstance. The number of positive samples in the matrix is determinable from a binomial distribution, as shown in FIG. 5C. The right-bottom graph in FIG. 5C shows the number of retests required for different arrangements of 4 positive samples and “x” marks 3 of the 4 positive samples. For example, if the 3 positive samples locate in the first two cells of the first row and the second cell of the second row, as shown in the top matrix of the right-bottom graph in FIG. 5C, the last positive sample may locate in any of the four shaded squares. There is 1/13 possibility that the last one locates in the 2,2 square, and “2,2” means under this circumstance, 2 row-pools and 2 column-pools will be tested positive under the initial test. Samples located at the 4 intersections of the 2 rows and the 2 columns are equivocally positive, therefore, 4 retests are required. Similarly, there is 4/13 possibility that the last one locates in the 2,3 square, and “2,3” means under this circumstance, 2 row-pools and 3 column-pools will be tested positive under the initial test. Samples located at the 6 intersections of the 2 rows and the 3 columns are equivocally positive, therefore, 6 retests are required. It should be appreciated that the right-bottom graph in FIG. 5C is an exemplary graphs showing several possible arrangements of four positive samples and the arrangements of four positive samples are not exhausted in the graph. However, unlike a 1D pool which is only positive/negative, a discrete probability is calculated for each possible number of positives.

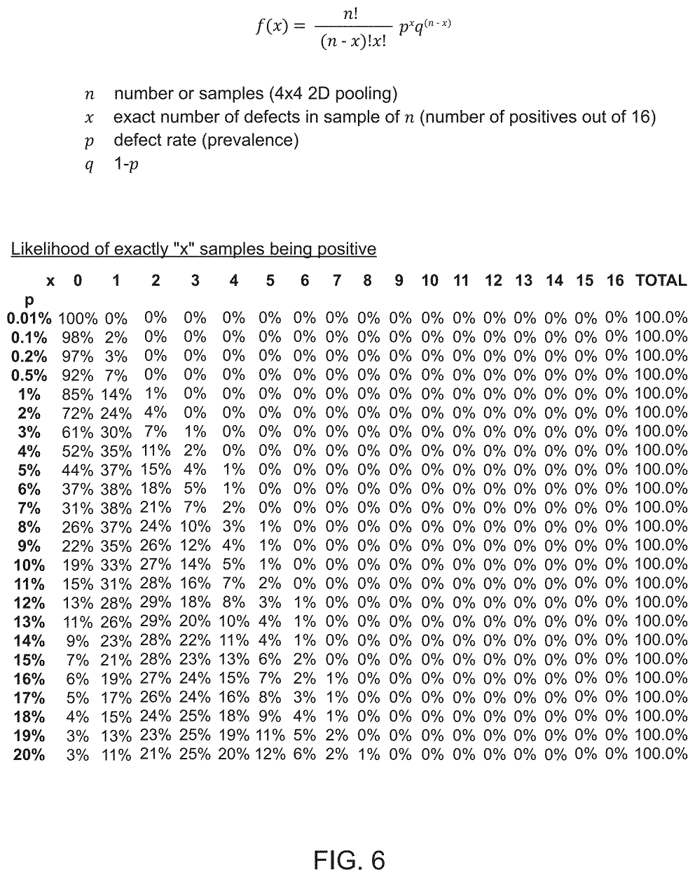

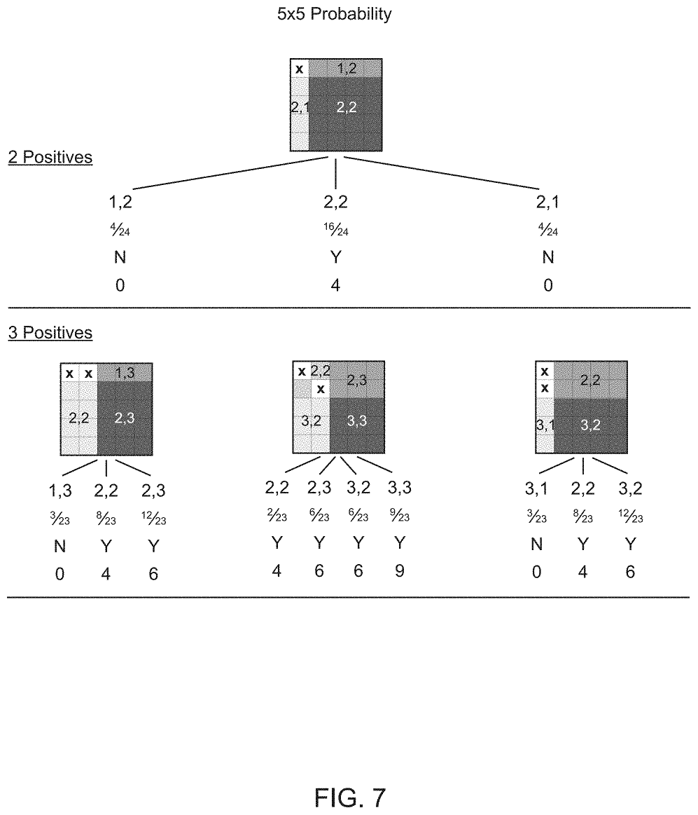

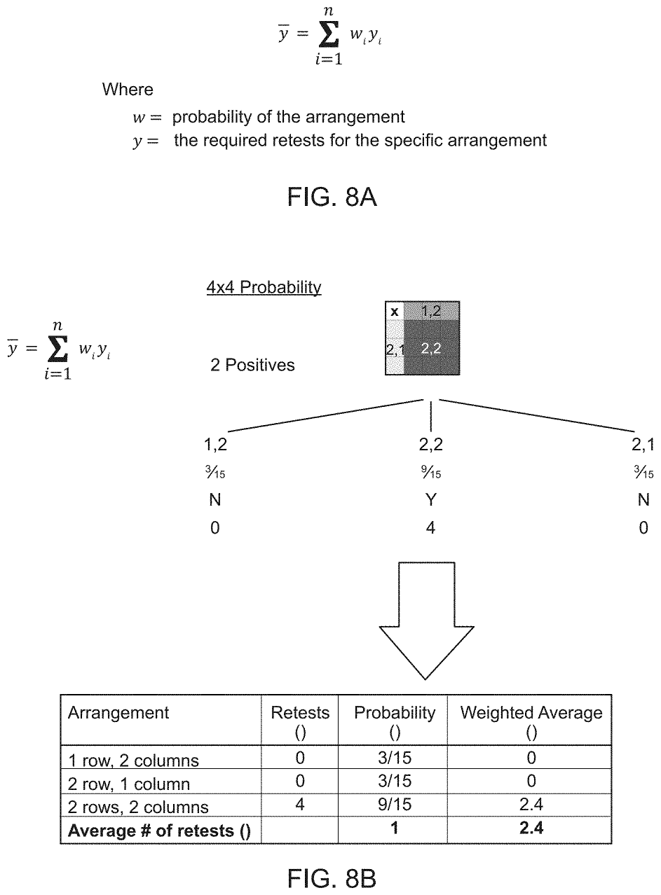

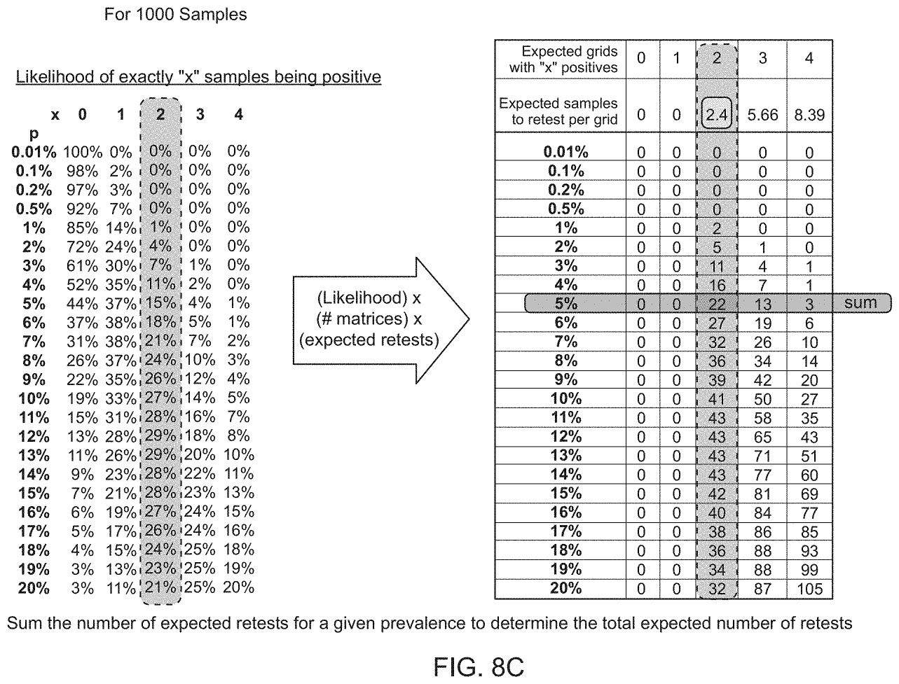

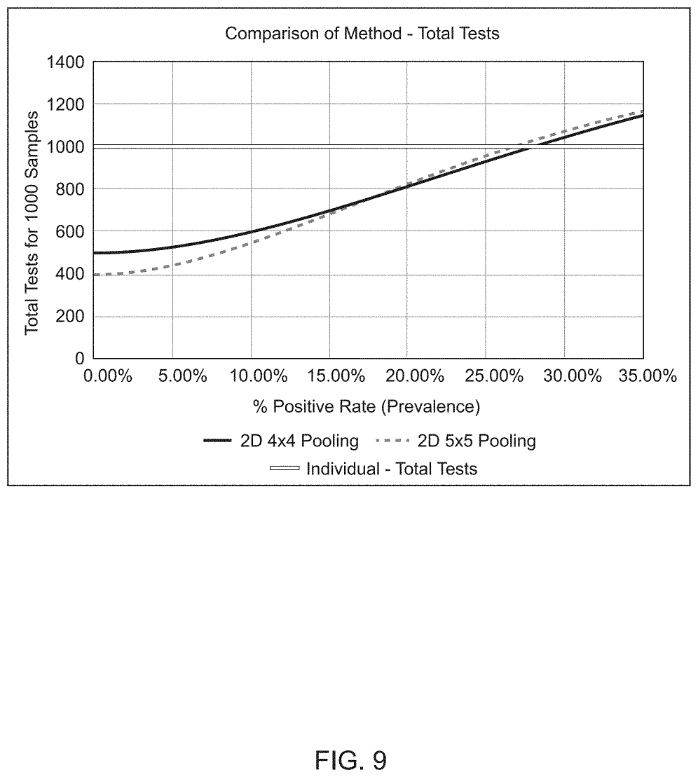

As shown in FIG. 6, a binomial distribution is calculated for the entire prevalence range to be analyzed—in this example between 0 and 20% prevalence. As shown in FIG. 7, the arrangement of positives within the matrix is determinable from a probability tree. For each arrangement of positive pools in the matrix, the number of retests required may be calculated from (#positive rows)×(#positive columns)=#retests (see, e.g., FIG. 3C). The probability of each discrete arrangement occurring within the matrix may be calculated within the probability tree. Therefore, for a given number of positives, there are n matrix arrangements, and the average number of retests required may be calculated, as shown in FIGS. 8A and 8B. The total number of expected retests can be obtained by summing the number of expected tests for a given prevalence, as shown in FIG. 8C. Accordingly, the predicted number of total test (#initial tests+#retests) can be calculated for a 2D pooling. FIG. 9 shows a comparison of total tests predicted for a 2D 4×4 pooling, a 2D 5×5 pooling, and an individual testing over a positivity rate of between 0 and 35%. As shown, the 2D 5×5 pooling performs better at lower positivity rates (i.e., <10%); whereas the 2D 4×4 pooling performs better between 10% and about 24% positivity rates; and beyond about a 24% positivity rate the individual testing outperforms both 2D 4×4 pooling and 2D 5×5 pooling.

In some embodiments, the design of the pooling system and/or method is developed based on at least one of: (1) the assay sensitivity and/or (2) the prevalence of the pathogen in the population to be tested. For example, and not in any way limiting, a pool size may depend on whether the assay is sensitive enough to detect the pathogen in a sample that has been diluted e.g., 1:2 (where 2 samples are pooled), or 1:3 (where 3 samples are pooled), or 1:5, or 1:10, or 1:125 (for a 5×5×5 three dimensional array), or 1:512 (for a 8×8×8 three dimensional array) or any other array formats. Additionally and/or alternatively, the pool size may depend on the prevalence of the pathogen in the testing population. If the pathogen is very rare (e.g., <1%), and the sensitivity is high, larger pools can be used. However, if the pathogen is fairly common (e.g., greater than 2-10%) a smaller pool size may be needed to reduce the number of positive samples per pool.

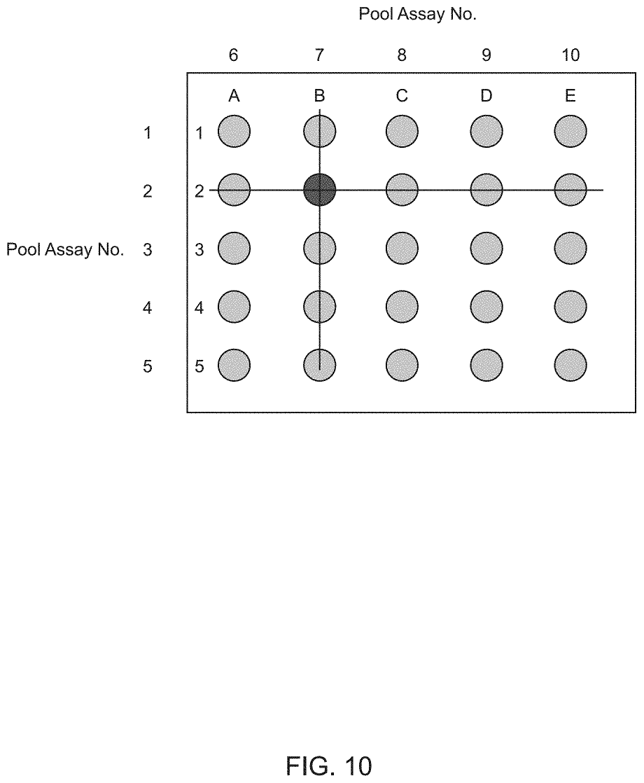

By pooling samples and using a two-dimensional or a three-dimensional grid, the time and number of tests required is significantly reduced without compromising test integrity. For example, as shown in FIG. 10, a 5×5 two-dimensional strategy allows for testing of 25 samples using only 10 assays (shown as 1-10 in FIG. 10). Upon detection of a positive sample, for example, for the sample included in pool assay numbers 2 and 7 (indicated by the line connecting pooled samples), it can be determined that the positive result corresponds to the sample at position B2. This positive result can be confirmed by retesting that particular sample. If only one-dimensional pooling was used, each of the samples in row 2 would have to be retested, thereby requiring another 5 tests for a total of 6 tests for 5 samples as compared to 11 tests for 25 samples.

In some embodiments, the design of the pooling system and/or method is developed based on: (1) the assay sensitivity and (2) the prevalence of the pathogen in the population to be tested. The design process may include selecting samples for aliquoting into the pooling system based at least in part on the origin of the sample (e.g., one or more regions or populations). Additionally and/or alternatively, selection of the samples for aliquoting into the pooling system is based at least in part on an expected disease prevalence. For example, samples may be grouped (i.e., pre-sorted prior to pooling) based on sample origin data such as, but not limited to, zip code or state. Or, samples may be sorted based upon other population demographics known to be associated with disease prevalence (e.g., specific communities, subject age, or travel history). Or, other factors associated with disease prevalence may be used.

Thus, the selection and sorting of samples (prior to pooling) can take account for expected prevalence of the disease in a particular region or population. For example, samples from a region exhibiting a very low prevalence of the disease in a population (e.g., <2%) may be included in the pool group that includes samples exhibiting a relatively high prevalence of the disease in the population (>10%) such that the expected prevalence of the positive samples is optimized for the pooling procedure used (e.g., disease prevalence of about 5%). Or samples from multiple regions may be included in the pool group. For example, the pool may include about 25% of the samples from a region of high disease prevalence (e.g., >10%), 25% of the samples from a region of low disease prevalence (<1%), and about 50% of the samples from a region of average disease prevalence (about 5%) such that the pooled samples have an expected disease prevalence that maximizes unequivocal identification of samples as either positive or negative for the pathogen of interest without the need for retesting. The prevalence of the pathogen in the pooled group may be obtained or estimated from historical and/or real-time records of positivity rate for the pathogen in the given region(s) and/or population(s).

Samples may be sorted at the site of procurement or in the laboratory performing the test. For example, in some cases samples are grouped at the site of procurement based on the subject's zip-code. Thus, samples from each zip-code may be pre-grouped at the procurement site for subsequent pooling at the testing lab. Or, in some cases samples may be pooled at the site of procurement and the pooled samples sent to a testing lab. For example, this can reduce shipping time and costs. In such cases, the original samples may be maintained at the site of procurement.

The design process may further include using testing of actual samples to identify a pooled testing protocol for the pooling system. Identifying the pooled testing protocol may comprise generating a plurality of potential multidimensional matrices (e.g., a 2D 4×4, a 2D 5×5, a 3D 5×5×2, etc.) for testing the multiple samples for the pathogen. Each potential multidimensional matrix provides for column, row, and/or address based pooling of the multiple samples, and a size of the potential multidimensional matrix is determined by a number of samples in the columns, rows, and/or addresses that is selected based on a sensitivity of the testing assay for the pathogen. Once the plurality of potential multidimensional matrices. For example, and not in any way limiting, a pool size or number of samples in the columns, rows, and/or addresses may depend on whether the assay is sensitive enough to detect the pathogen in a sample that has been diluted e.g., 1:2 (where 2 samples are pooled), or 1:3 (where 3 samples are pooled), or 1:5, or 1:10, or 1:125 (for a 5×5×5 three dimensional array), or 1:512 (for an 8×8×8 three dimensional array) or any other array formats. Additionally and/or alternatively, the pool size or number of samples in the columns, rows, and/or addresses may depend on the prevalence of the pathogen in the testing population. If the pathogen is very rare (e.g., <1%), and the sensitivity is high, larger pools can be used. However, if the pathogen is fairly common (e.g., greater than 2-10%) a smaller pool size may be needed to reduce the number of positive samples per pool.

For each potential multidimensional matrix a number of initial test assays to be performed is determined based on the column, row, and/or address based pooling of the multiple samples and the size of the potential multidimensional matrix. Additionally, for each potential multidimensional matrix a number of retest assays to be performed is predicted based on a predicted number of positive samples in the potential multidimensional matrix and a predicted arrangement of the positives within the potential multidimensional matrix. The predicted number of positive samples is determined based on a discrete probability calculated for each possible number of positives based on the prevalence of the pathogen in the population to be tested, and the predicted arrangement of the positives is determined based on a discrete probability calculated for each possible positive arrangement occurring within the potential multidimensional matrix. For each potential multidimensional matrix a total number of test assays to be performed is predicted based on the number of initial test assays and the number of retest assays.

Identifying the pooled testing protocol may further comprise comparing the predicted total number of test assays to be performed for each potential multidimensional matrix against the predicted total number of test assays to be performed for all other potential multidimensional matrices within the plurality of potential multidimensional matrices. The potential multidimensional matrix that satisfies a given criteria (e.g., the matrix with the least total number of test assays to be performed) based on the comparison as a multidimensional matrix is selected to form a basis for the pooled testing protocol and used in the pooling system. Once the pooled testing protocol is identified, the multiple samples are aliquot in the multidimensional matrix (which can be a physical or a virtual matrix) based on the pooled testing protocol, and samples from each column, row, and/or address of the multidimensional matrix are pooled. As noted above, the aliquoting may be done at the collection site, the testing site or anywhere in between. The pooled samples may be tested with the testing assay to determine a presence or absence of a detectable amount of the pathogen in each of the pooled samples, and based on the presence or absence of the detectable amount of the pathogen in each of the pooled samples, a determination is made as to whether at least one individual sample comprises the detectable amount of the pathogen. In certain instances, the at least one individual sample that comprises the detectable amount of the pathogen is identified as an unequivocal sample that is common to a row and column or a row, column, and address of pooled samples that each comprise a detectable amount of the pathogen. In some instances, individual samples identified as equivocally positive or potentially positive for comprising a detectable amount of the pathogen are retested with the test assay.

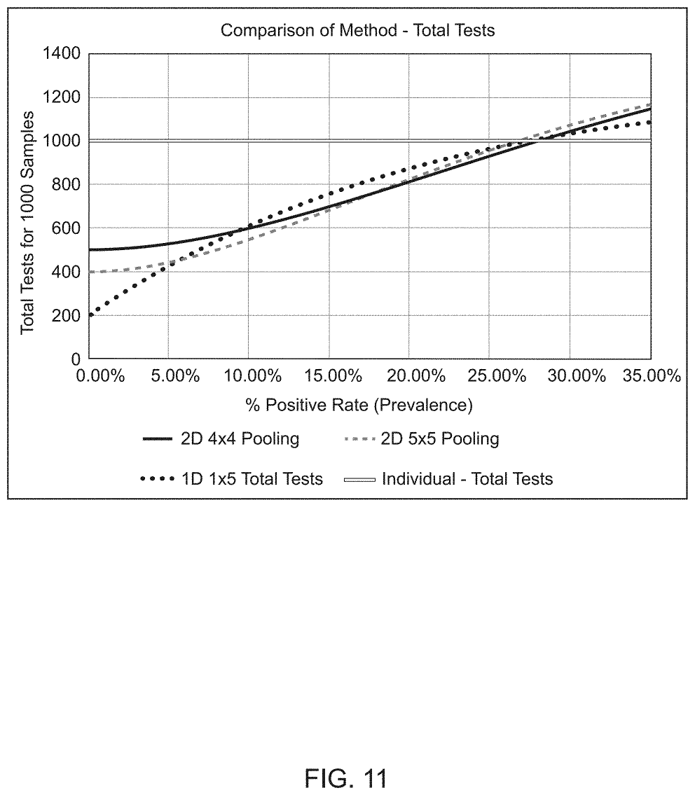

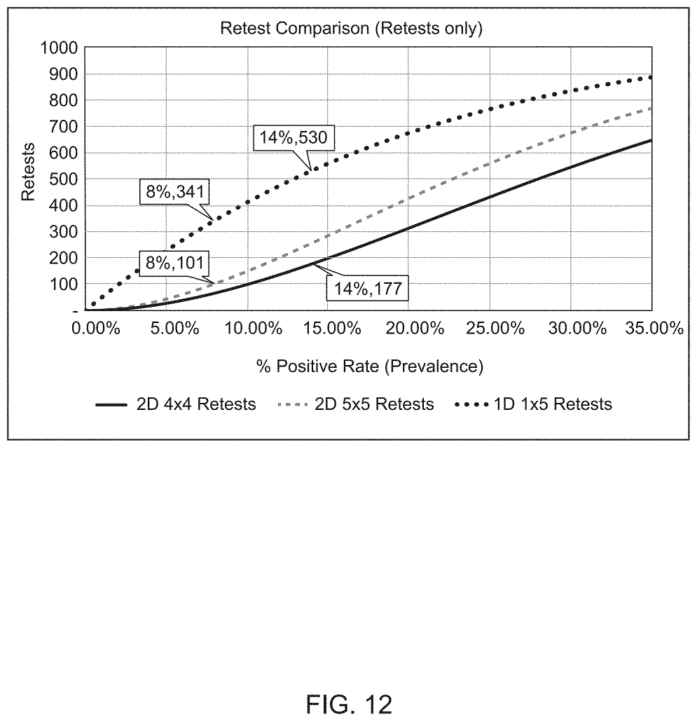

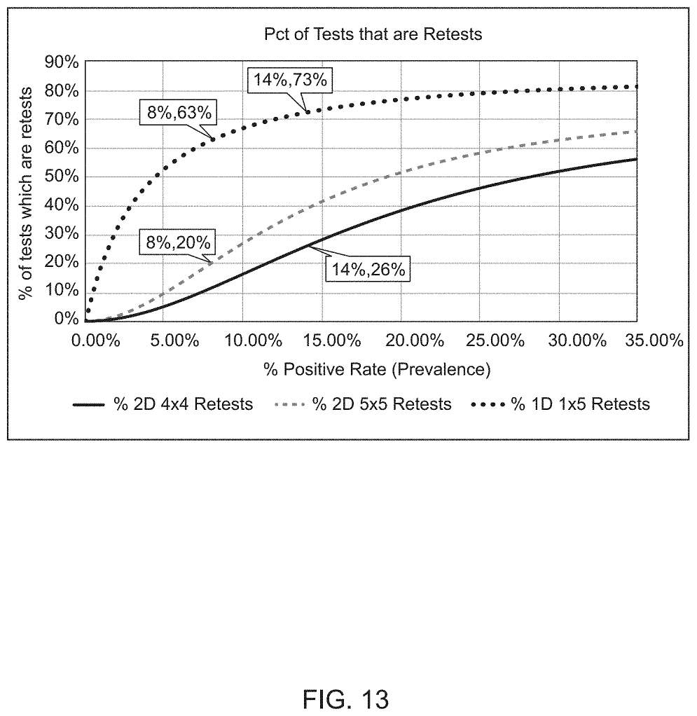

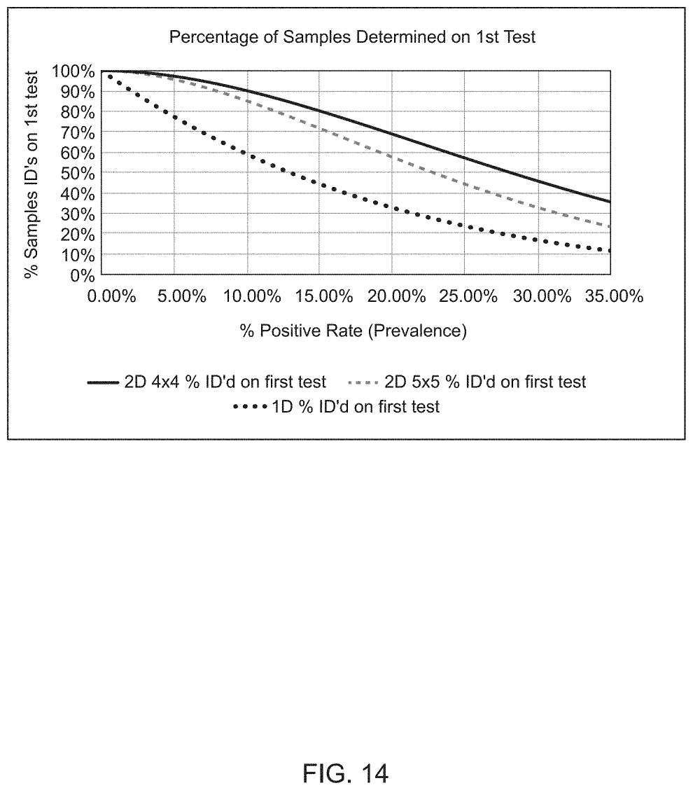

FIGS. 11-14 illustrate benefits of reduced total tests and retests achievable using pooled testing protocols designed in accordance with the various embodiments described herein. FIG. 11 shows expected total tests to 1000 samples using individual testing as compared to multiple pooled testing protocols including a 1D pooling of 5 samples, 2D 4×4 matrix pooling, and 2D 5×5 matrix pooling. In general, all pooled testing protocols are predicted to require fewer total tests than individual testing below certain prevalence levels (e.g., <about 28%) (x-axis). Above these prevalence levels, individual testing may require fewer tests than the pooled testing protocols. FIG. 12 shows a comparison of the number of retests required for 1D pooling of 5 samples as compared to 2D 4×4 pooling or 2D 5×5 pooling. It can be seen that in an embodiment, both 4×4 and 5×5 two dimensional matrix pooling require significantly fewer retests to provide unequivocal results than one dimensional 1×5 pooling. FIG. 13 shows the percentage of tests that are retests for different pooling methods for a given disease prevalence. It can be seen that in an embodiment, both 2D 4×4 pooling or 2D 5×5 pooling require a significantly lower proportion of retests compared to 1D pooling of 5 samples. FIG. 14 shows a comparison of the percentage of samples that are unequivocally identified (as positive or negative) on the first test for different pooling methods for a given prevalence. It can be seen that in various embodiments, both 2D 4×4 pooling and 2D 5×5 pooling provide a significantly higher percentage of unequivocal results on the first test as compared to 1D pooling of 5 samples.

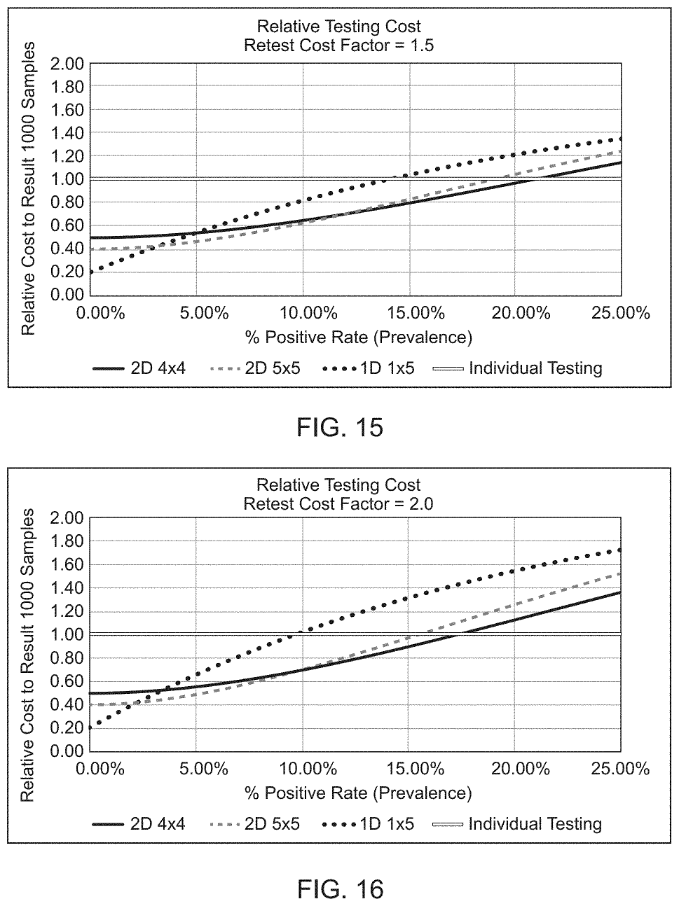

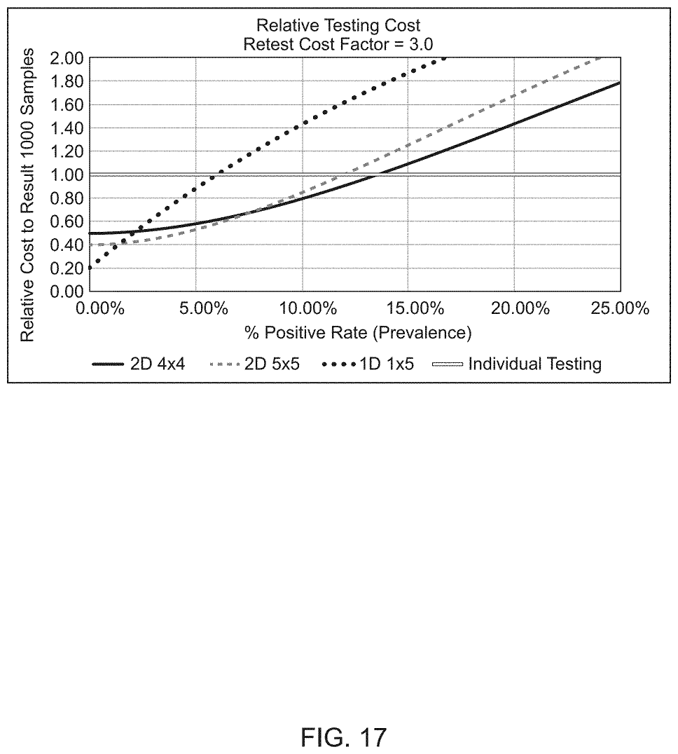

FIGS. 15-17 illustrate benefits of cost savings achievable using pooled testing protocols designed in accordance with the various embodiments described herein. Due to the archiving and retrieval process, retesting is often more costly than initial testing. A cost factor (CF) can be applied to the retest numbers to quantify and compare the testing methodologies. This cost factor is always greater than 1. For example, a cost factor of 2.0 would indicate a retest is twice as costly to perform as an initial test. This could include a combination of hard costs, such as the expense with retrieving archive samples, and soft costs, such as the increase in turnaround time to provide results due to retesting. In an embodiment, incremental cost for retesting is measured from the point in the workflow that an individual sample is aliquot in the lab to the point where it is resulted. The cost factor can never be less than one, and will generally be higher than one due the additional labor required to reintroduce an archived sample into the workflow. The analysis was performed on 1,000 samples for the indicated testing methods. As shown in FIGS. 15-17, it can be seen that for a cost factor greater than 1.0, there are significant cost benefits to 2D matrix pooling 4×4 and 5×5 over both 1D 1×5 and individual testing when a cost factor is applied. In this example, these benefits accrue in the 2-18% prevalence range, depending on the cost factor.

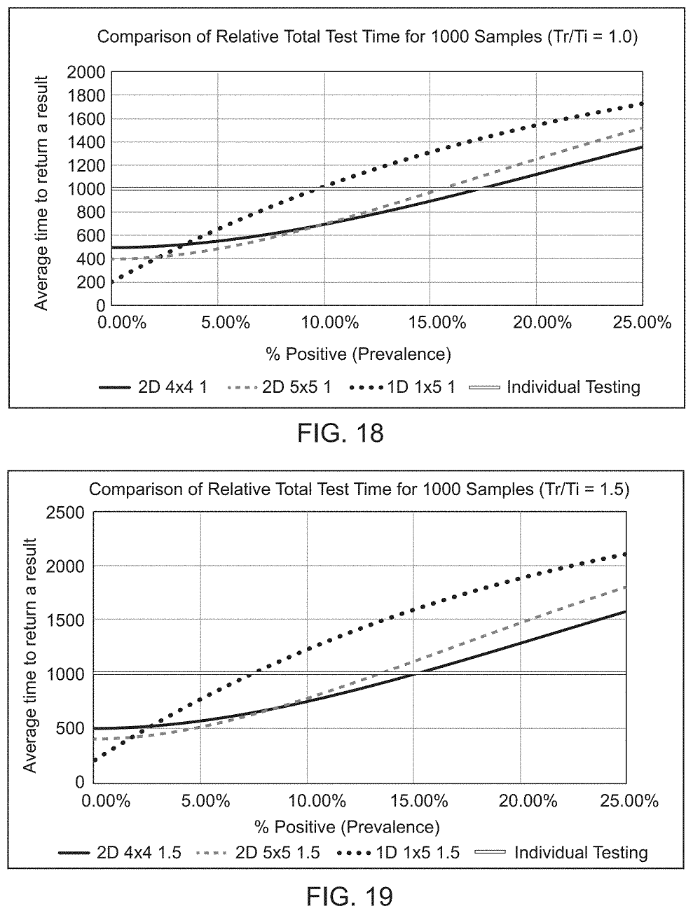

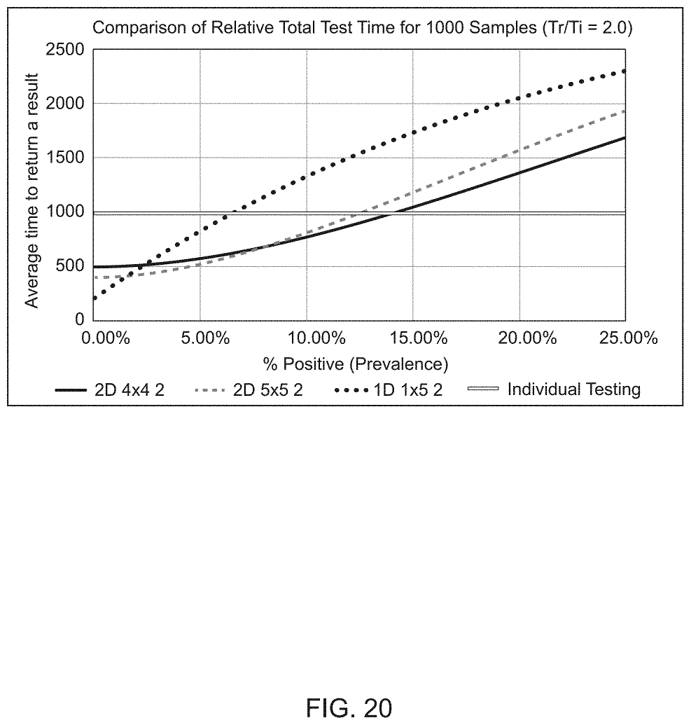

FIGS. 18-20 illustrate benefits of time savings achievable using pooled testing protocols designed in accordance with the various embodiments described herein. The overall turnaround time to provide a result for a sample requiring a retest will always be longer than a sample requiring only an initial test. Thus, minimizing retests is essential to reducing overall testing turnaround time. Total Test Time can be calculates as Ti+Tr, where Ti=Initial Test Time and Tr=Retest Test Time (Tr>=Ti). As shown in FIGS. 18-20, it can be seen that for any retesting, there are significant time savings to both 2D matrix pooling 4×4 and 2D matrix pooling 5×5 over 1D 1×5 when a retesting factor of 1.0 or more is applied. In this example, these benefits accrue in the 2-25% prevalence range, depending on the retesting factor.

Thus, the pooled testing protocols designed in accordance with the various embodiments save significant time and resources. This can be extremely important when results are need quickly and tests are being run at a high volume. For example, in an embodiment, the methods and systems of the disclosure are applied to COVID-19 viral testing. Thus, in an embodiment, the method comprises real-time reverse transcription polymerase chain reaction (rRT-PCR) as described in co-pending Provisional Patent Application 63/004,143, filed Apr. 2, 2020 and entitled Methods and Systems for Detection of COVID-19, which is incorporated by reference in its entirety herein. In an embodiment, the test uses three primer and probe sets to detect three regions in the SARS-CoV-2 nucleocapsid (N) gene (e.g., N1, N2 and N3) and one primer and probe set to detect human RNase P (RP) in a clinical sample.

A variety of sample types may be used. In an embodiment, RNA is isolated from upper and lower respiratory specimens. Such specimens (samples) may include nasopharyngeal or oropharyngeal swabs, sputum, lower respiratory tract aspirates, bronchoalveolar lavage, and nasopharyngeal wash/aspirate or nasal aspirate). The RNA may then be reverse transcribed to cDNA and subsequently amplified using quantitative PCR. In an embodiment, the RT-PCR comprises a multiplex reaction with the COVID-19 primers and probes. In other embodiments, the RT-PCR comprises a multiplex reaction with the COVID-19 primers and probes and the RP primers and probes.

Also disclosed are systems for performing the methods herein. For example, the system may comprise a station or stations for performing various steps of the methods. For example, a system may comprise a matrix with samples aliquoted in rows and columns and/or a plurality of 2D matrices arranged in 3D format. Or, the system may comprise a component for defining a virtual matrix with samples assigned to rows and columns and/or a plurality of 2D matrices arranged in 3D format. The system may comprise a station for preparing the matrices. Additionally and/or alternatively, the system may comprise a station for running the test (i.e., determining if a pool or a sample comprises a detectable amount of the pathogen). Additionally, the system may comprise a station for analyzing the results of the test for each of the pools and determining which individual sample or samples is positive. In certain embodiments, a station may comprise a robotic station for performing the step or steps. Additionally, the system may comprise a computer-program product tangibly embodied in a non-transitory machine-readable storage medium, including instructions configured to run the systems and/or perform a step or steps of the methods of any of the disclosed embodiments.

Thus, also disclosed is a computer-program product tangibly embodied in a non-transitory machine-readable storage medium, including instructions configured to run the systems and/or perform a step or steps of the methods of any of the disclosed embodiments. In one embodiment, the system comprises a computer-program product tangibly embodied in a non-transitory machine-readable storage medium, including instructions configured to determine the optimal number and array system, e.g., 2D or 3D, and/or the number of samples pooled in each dimension. Additionally and/or alternatively, the computer program product may comprise instructions for forming a matrix with samples aliquot in rows and columns and/or a plurality of 2D matrices arranged in 3D format. Or, the computer program product may comprise instructions for defining a virtual matrix with samples assigned to rows and columns and/or a plurality of 2D matrices arranged in 3D format. As noted above, this may depend on the sensitivity of the assay and/or the prevalence of the pathogen in the population.

II.B. Non-Square Pooling Techniques for Minimizing Retesting

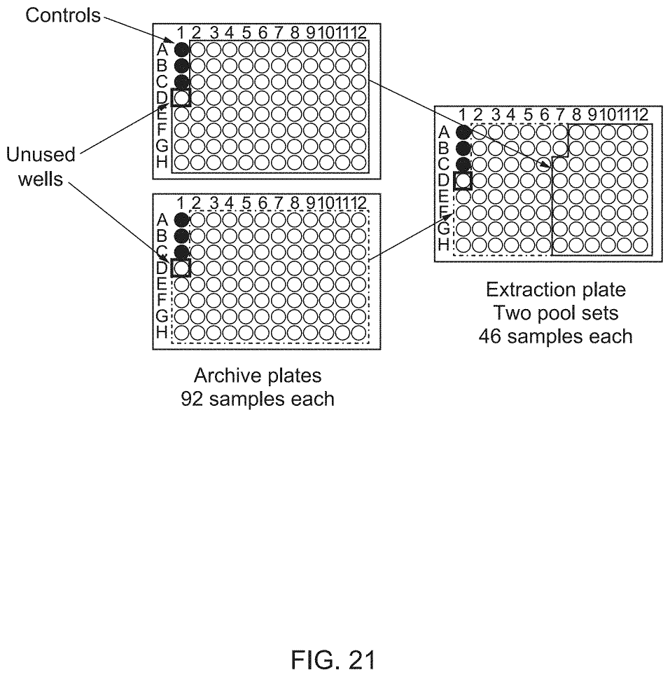

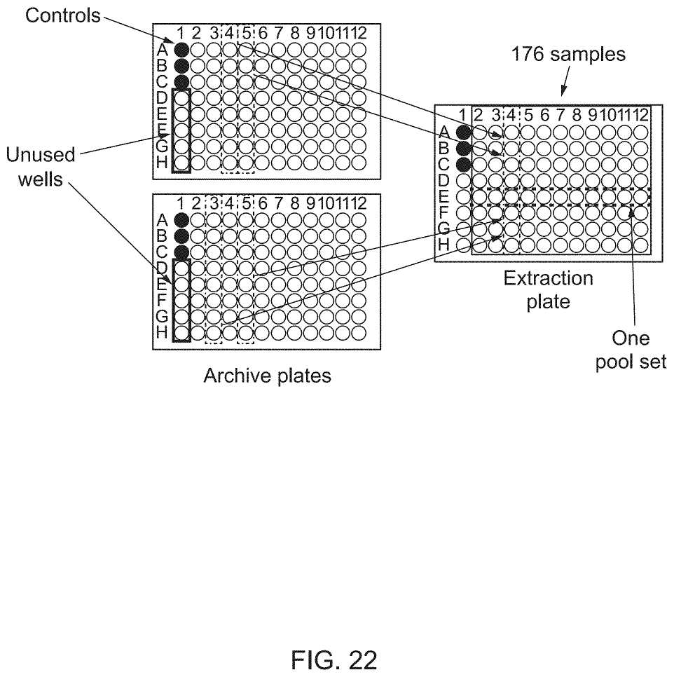

In some instances, the method for performing high-throughput testing for a pathogen may utilize non-square pooling techniques. For example, FIG. 21 illustrates an exemplary instance where non-square pooling techniques are advantageous. In FIG. 21, only 92 real-time samples with 3 control samples and one unused well are on each 96 well plate. In this instance, a square matrix pooling method may not be a best choice, and thus a non-square pooling technique is beneficial. In some embodiments, each plate with 92 real-time samples may be organized into two pool sets, where each pool set contains 46 samples. FIG. 22 illustrates another instance where non-square pooling techniques are beneficial. In FIG. 22, there are three control samples and five unused wells on each 96 well plate. When two of such 96 well plates are combined together, there are 176 samples in total located on a 8×11 virtual matrix. The rows may be pooled together from the matrix to form pool sets, for example, there are 22 samples in each row resulting in 22 samples in each pool set. However, the size of the pool sets makes it improper to perform a squared matrix pooling. Therefore, non-square pooling techniques are desired.

A non-square pooling technique is a matrix pooling technique where numbers of rows columns, and/or address are different. A non-square pooling technique may be a 1D pooling technique. A non-square pooling technique may also be a double/triple/multiple pooling technique.

A double pooling technique is designed to pool and test multiple samples in a plurality of pools where each pair of pools overlaps in at most a predetermined number of samples and where each sample is in exactly two pools. In various embodiments, a number of pools is determined based on a prevalence or positive rate for a pathogen being detected by the pooled testing. In some embodiments, a number of pools is determined based on a sensitivity of a test assay. In some embodiments, a number of pools is determined based on both a prevalence or positive rate for a pathogen being detected by the pooled testing and a sensitivity of a test assay. It should be appreciated that a number of pools in double pooling techniques may be based on other variables that are known to an ordinary person with skilled in the art. In various embodiments, a size of each pool is the same in a double pooling technique. In other embodiments, a size of each pool is different or varies in a double pooling technique.

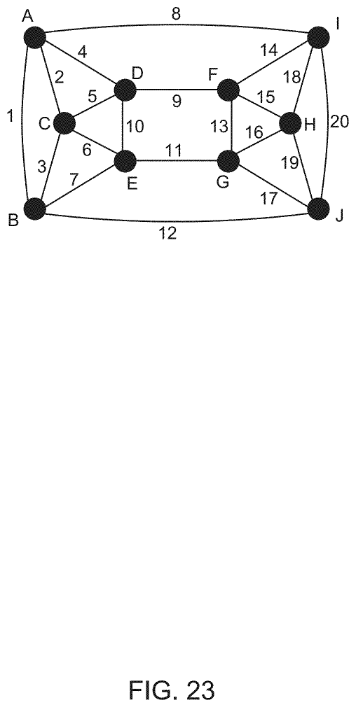

In some embodiments, each pair of pools in a double pooling technique overlaps in at most one sample. FIG. 23 illustrates a double pooling design with 10 pools of size 4 using a graph with 10 vertices and 20 edges. Each vertex (A-J) represents a pool and each edge (1-20) represents a sample. For example, Pool D contains Samples 4, 5, 9 and 10, and Pool F contains Samples 9, 13,14, and 15. It is illustrated from the graph that each vertex has four edges connected to it, therefore, each pool has four samples to be tested. Because each edge connects exactly two vertices, the graph design also shows a double pooling technique where each pair of pools overlaps in at most one sample and where each sample is in exactly two pools. For example, Pools D and F share only one sample (Sample 9) because the corresponding edge connects D and F, and Pools D and H share no sample because there is no direct connection between D and F. When performing a double pooling design shown in FIG. 23, corresponding samples to the four edges connected to each vertex form one pool and are pooled and tested according to a corresponding pooled testing protocol.

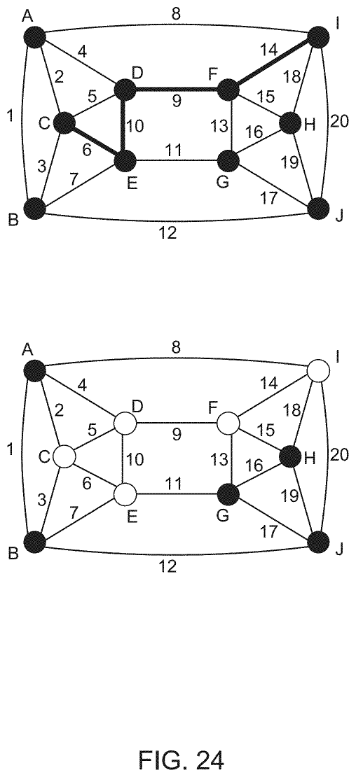

A double pooling technique may yield unequivocally positive results or equivocally positive results. FIG. 24 shows one instance where a double pooling design with 10 pools of size 4 yields both unequivocally positive results and equivocally positive results. The top graph in FIG. 24 shows Samples 6, 9, 10, and 14 are positive samples. When using the double pooling design introduced in FIG. 23 and the 10 Pools A-J are pooled and tested, Pools C, D, E, F, and I should be tested positive because each pool contains at least one positive sample, as shown in the bottom graph of FIG. 24.

Not all individual samples in each positive pool are required for retest. For example, of the four samples 4, 5, 9, and 10 in Pool D, Sample 4 is unequivocally negative because Pool A also contains Sample 4 and Pool A is negative. Similarly, Samples 2, 3, 7, 8, 11, 13, 15, 18, and 20 are all unequivocally negative. Moreover, Sample 14 is unequivocally positive because Pool I is positive, whereas all other pools in which the other three samples are pooled are negative. Therefore, Samples 2-4, 7-8, 11, 13-15, 18, and 20 need not to be retested because they are unequivocal samples, and Samples 1, 12, 16-17, and 19 need not to be retested because they are not in any positive pools. However, all other samples (Sample 5, 6, 9, and 10) need to be retested because they are equivocally positive. It is possible that all four of Sample 5, 6, 9, and 10 are positive, it is possible that Samples 5 and 6 are positive while Samples 9 and 10 are negative, and it is also possible that other combinations of positive samples yield the same positive pools.

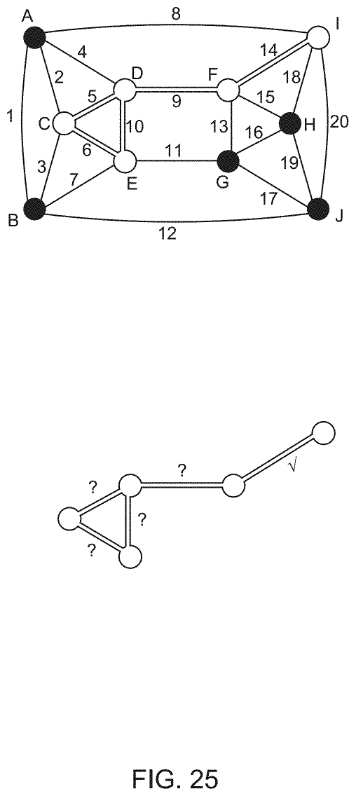

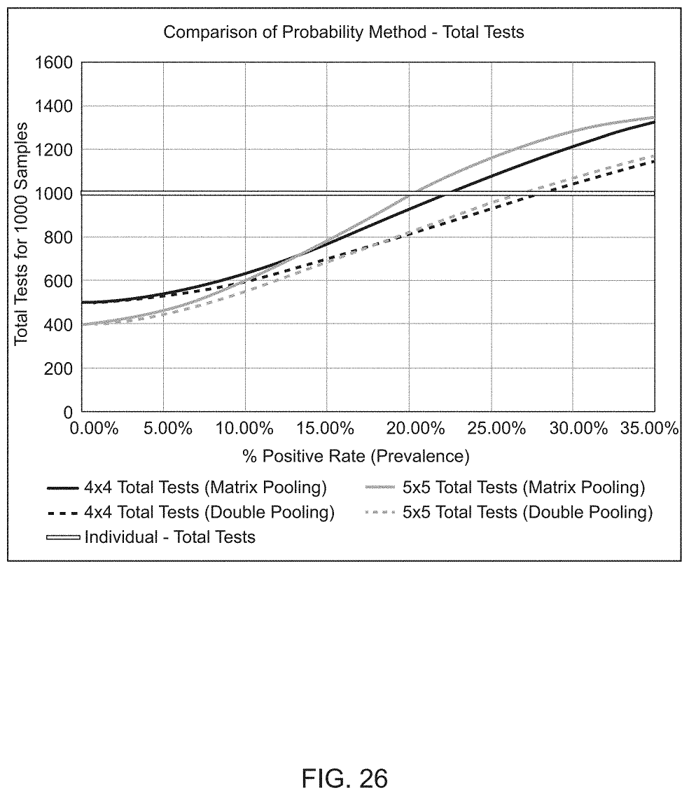

To solve the problem of figuring out which sample(s) need to be retested, a subgraph can be constructed in silico by connecting all positive vertices together, as shown in FIG. 25. Only samples (edges) in the subgraph could be a candidate to be retested. A technique can help further limit the retest size. As shown in the bottom graph of FIG. 25, if a vertex (here Vertex I) connects to just one other vertex (Vertex F), then the edge connected to these two vertex must correspond to a positive sample. This technique can help limit the number of retests efficiently. FIG. 26 shows a comparison of total test numbers among different pooling techniques. Specifically, FIG. 26 shows that the double pooling with the technique shown in FIG. 25 substantially decreases the number of total tests, and both a 4×4 double pooling technique and a 5×5 double pooling technique performs better than an individual testing technique when a prevalence is under 25%-28%.

It should be appreciated that a triple or multiple pooling technique can be performed using the similar methods as disclosed above. For example, a triple pooling technique may be designed to pool and test multiple samples in a plurality of pools where each pair of pools overlaps in at most a predetermined number of samples and each sample is in exactly three pools. Further, a multiple pooling technique may be designed to pool and test multiple samples in a plurality of pools where each pair of pools overlaps in at most a predetermined number of samples and each sample is in exactly a certain number of pools. In some embodiments, one or more samples may be designed to be in a different number of pools than a number of pools another one or more samples is within.

II.C. Intelligent Sample Selecting Techniques

The choice of pooling techniques for a pathogen depends on a prevalence of the pathogen in a sample set to be tested. For example, with a relatively high prevalence (e.g., a prevalence of greater than 30%), individual testing may be more efficient; and with a relatively low prevalence, a 4×4 matrix pooling may be more efficient. A challenge faced in choosing pooling techniques is how to select and combine samples from different demographic locations to perform the most efficient testing technique.

Techniques for intelligently selecting samples to perform a pooled testing for a pathogen is desired to solve the sample selection and combination challenge. In various embodiments, a method comprises obtaining samples from a plurality of regions or populations, where the samples from each region or population form a sample selection candidate set; determining a prevalence of the pathogen in the samples from each region or population of the plurality of regions or populations; determining, by an intelligent selection machine, an optimal selection plan to perform the pooled testing on the samples, where the optimal selection plan comprises an optimal ratio to combine the samples from the plurality of regions or populations, an optimal prevalence in a combined sample set, and an optimal pooling design for the pooled testing; selecting samples from one or more sample selection candidate set based on the optimal ratio; combining the selected samples to form the combined sample set with the optimal prevalence; aliquoting the samples in the combined sample set based on the optimal pooling design; pooling the samples in the combined sample set based on the optimal pooling design; testing the pooled samples to determine a presence or absence of a detectable amount of the pathogen in each of the pooled samples; and determining, based on the presence or absence of the detectable amount of the pathogen in each of the pooled samples, whether at least one individual sample comprises the detectable amount of the pathogen.

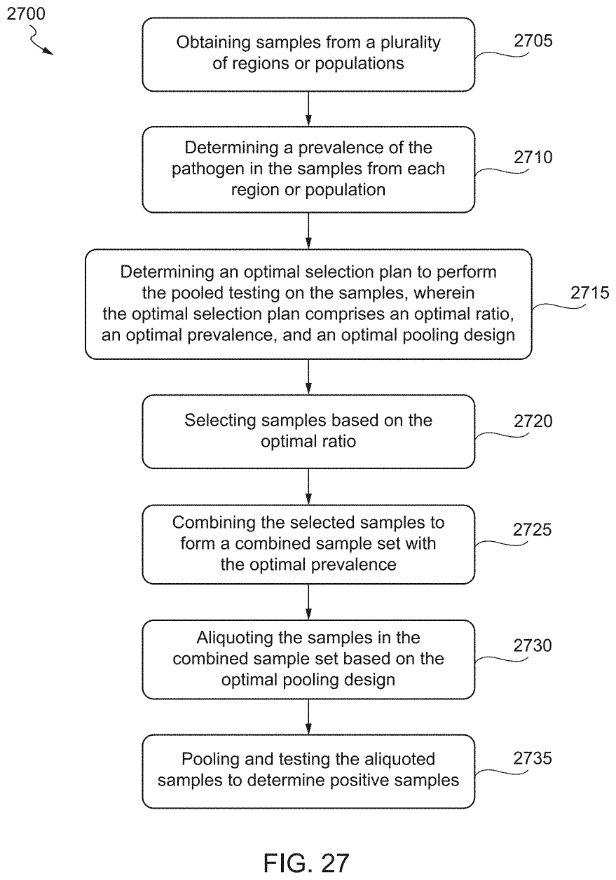

FIG. 27 is a flowchart illustrating a process 2700 for performing intelligent sample selection and pooled testing according to various embodiments. The processing depicted in FIG. 27 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 27 and described below is intended to be illustrative and non-limiting. Although FIG. 27 depicts the various processing steps occurring in a particular sequence or order, this is not intended to be limiting. In certain alternative embodiments, the steps may be performed in some different order or some steps may also be performed in parallel. In certain embodiments, the processing or a portion of the processing depicted in FIG. 27 may be performed by a computing device such as a computer (e.g., the intelligent selection machine).

At block 2705, samples to be tested are obtained. Because the intelligent sample selection techniques are preferable techniques to combine samples from different regions or populations to achieve a desired prevalence of a pathogen, the samples are generally obtained from different regions or populations. In various embodiments, samples are obtained based on regions or populations. In some embodiments, samples are obtained based on demographic information such as zip code, age, vaccination status and/or countries recently visited. It should be appreciated that samples may simply be obtained or collected from different collection sites and pre-analyzed and grouped according to regions, populations, or other demographic information. Samples may be obtained and grouped into different sample selection candidate sets based on regions, populations, or other demographic information. In some embodiments, samples obtained at block 2705 comprise a specimen from either an upper or lower respiratory system. In some embodiments, samples obtained at block 2705 comprise at least one of a nasopharyngeal swab, an oropharyngeal swab, sputum, a lower respiratory tract aspirate, a bronchoalveolar lavage, a nasopharyngeal wash and/or aspirate or a nasal aspirate. In some embodiments, the pathogen is SARS-CoV-2.

At block 2710, a prevalence of the pathogen in each sample selection candidate set is determined. In various embodiments, the determination of the prevalence of the pathogen may be based on a historical record. In some embodiments, the determination of the prevalence of the pathogen may be based a real-time data. It should be appreciated that any method that is reliable and relatively stable can be used to determine the prevalence of the pathogen. In some instances, the determination of the prevalence comprises obtaining the prevalence of information for calculating the prevalence from an external source such as a government agency reporting. In some instances, the determination of the prevalence comprises obtaining the prevalence from internal testing and reporting of prior samples from similar or same regions or populations. In some instances, a combination of internal and external data is used to determine the prevalence of the pathogen.

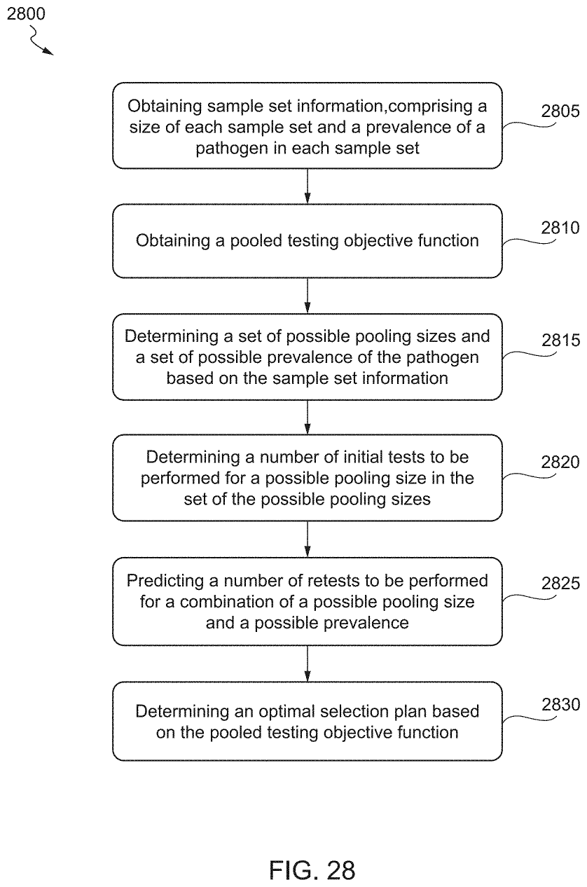

At block 2715, an optimal selection plan to perform the pooled testing on the samples is determined, where the optimal selection plan comprises an optimal ratio to combine the samples from the plurality of regions or populations, an optimal prevalence in a combined sample set, and an optimal pooling design for the pooled testing. In various embodiments, the optimal selection plan is determined by an intelligent selection machine (i.e., a specialized computing device). The intelligent selection machine is explained in further detail with respect to FIG. 28. As used herein, “optimal” means the “best possible” or “most favorable.”

At block 2720, samples are selected from one or more sample selection candidate sets based on an optimal ratio. For example, if there are two sample selection candidate sets A and B, a prevalence in Set A is 2%, and a prevalence in Set B is 10%. If the optimal ratio determined at block 2715 is 1:1, then 50% of samples in a pool set is selected from Set A and 50% from Set B. A prevalence of the pool set thus is 6%. In various embodiments, an optimal prevalence is linked to the optimal ratio. In such instances, an optimal prevalence determined at block 2715 should be 6%. In some embodiments, an optimal ratio is linked to a plurality of optimal prevalence. For example, if the number of samples in Set A is triple the number of samples in Set B, there are samples from Set A to be unselected. The unselected or remaining samples in Set A are selected automatically and constitute another pool set with an optimal prevalence of 2%. It should be appreciated that an optimal ratio can be linked to more than two optimal prevalence when a number of the sample selection candidate sets is greater than two. In some embodiments, an optimal plan determined at block 2715 comprises multiple optimal ratios corresponding to multiple optimal prevalence, and samples are selected at block 2720 based on the multiple optimal ratios corresponding to the multiple optimal prevalence to form multiple pool sets. It should be appreciated that a ratio or an optimal ratio is not limited to a relationship between two sets and it may refer to a relationship among three or more sets. For example, a ratio or an optimal ratio among Sets A, B, and C may be 1:1:3 respectively, thus 20% of samples in a pool set is selected from Set A, 20% from Set B, and 60% from Set C. In various embodiments, samples are randomly selected from sample selection candidate sets. In some embodiments, samples are selected according to their indicia.

At block 2725, selected samples are combined to form a combined sample set (or a pool set) to be prepared to perform a pooled test. As mentioned above, samples selected based on an optimal ratio generally yield an optimal prevalence in the pool set. Therefore, a prevalence of the combined sample set is equal to an optimal prevalence, where the optimal prevalence may be determined by an intelligence selection machine, or the optimal prevalence may equal to a prevalence in a sample selection candidate set.

At block 2730, samples in a combined sample set or a pool set are aliquoted according to an optimal plan. In various embodiments, the aliquoting is based on the optimal pooling design determined at block 2715. For example, an optimal pooling design may be a 5×5 matrix pooling. Correspondingly, there should be 25 samples in each combined sample set, and the 25 samples are aliquoted in a 5×5 matrix in this instance. In some embodiments, the matrix is a physical array of the samples. In other embodiments, the matrix is an in silico array of the samples. The optimal pooling design is not necessarily a matrix pooling. A double pooling, a triple pooling, or a non-square pooling technique is also a suitable design to perform the aliquoting the samples. It should be appreciated that samples are not necessarily aliquoted into a matrix even under a matrix pooling design. It is practical to use other pooling techniques such as a double pooling technique to perform the aliquoting.

At block 2735, aliquoted samples are pooled and tested according to various embodiments. The testing may be performed with a testing assay to determine a presence or absence of a detectable amount of the pathogen in each of the pooled samples. Test results are used to determine positive samples in the samples obtained at block 2705. In some embodiments, a retest is needed to resolve or determine an equivocal positive sample. The pooling and testing at block 2735 may be performed by matrix pooling, double pooling, triple pooling, or non-square pooling techniques according to an optimal pooling design.

In various embodiments, an intelligent selection machine is configured to perform obtaining sample set information, where the sample set information comprises a size of each sample set and a prevalence of a pathogen in each sample set; obtaining a pooled testing objective function; determining a set of possible pooling sizes and a set of possible prevalence of the pathogen based on the sample set information; determining a number of initial tests to be performed for a possible pooling size in the set of the possible pooling sizes; predicting a number of retests to be performed for a combination of a possible pooling size in the set of the possible pooling sizes and a possible prevalence in the set of the possible prevalence; and determining an optimal selection plan based on the pooled testing objective function, where the optimal selection plan comprises an optimal ratio to combine samples in one or more sample sets, an optimal prevalence in a combined sample set, and an optimal pooling design for the pooled testing. It should be appreciated that an intelligent selection machine is not required to perform all functions introduced above, and not required to be configured to performed functions in the order above. An intelligent selection machine is designed to generate and provide information such as an optimal ratio to combine samples in one or more sample sets, an optimal prevalence in a combined sample set, and/or an optimal pooling design for performing a pooled testing.

FIG. 28 is a flowchart illustrating a process 2800 for performing functions configured in an intelligent selection machine according to various embodiments. The processing depicted in FIG. 28 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 28 and described below is intended to be illustrative and non-limiting. Although FIG. 28 depicts the various processing steps occurring in a particular sequence or order, this is not intended to be limiting. In certain alternative embodiments, the steps may be performed in some different order or some steps may also be performed in parallel. In certain embodiments, the processing depicted in FIG. 28 may be performed by a computing device such as a computer (e.g., the intelligent selection machine).

At block 2805, sample set information is obtained, where the sample set information comprises a size of each sample set and a prevalence of a pathogen in each sample set. In various embodiments, the sample set is a sample selection candidate set obtained at block 2705 in a process 2700. In some embodiments, the obtaining process at block 2805 may comprise counting a number of samples in each sample set to determine the size of each sample set. In various embodiments, the prevalence of the pathogen is obtained at block 2710 in a process 2700. In some embodiments, the prevalence of the pathogen is obtained independently.

At block 2810, a pooled testing objective function is obtained. The pooled testing objective function is used in subsequent steps of process 2800 to determine an optimal selection plan. The pooled testing objective function may be (i) a function to minimize a number of total tests, (ii) a function to minimize a number of retests, or (iii) a function to minimize a total cost of testing. It should be appreciated that the pooled testing objective function is not necessarily related to numbers of tests or retest, or a cost. The pooled testing objective function may be a multivariable determination function that takes different information such as sensitivity, specificity, and capacity of a test assay, or demographic information into consideration.

At block 2815, a set of possible pooling sizes and a set of possible prevalence of the pathogen is determined based on the sample set information. The set of the possible pooling sizes is determined based on (i) a sensitivity of a testing assay, (ii) a specification of a testing assay, (iii) the prevalence of the pathogen, (iv) a policy requirement, or (v) any combination thereof. For example, a policy may mandate that a number of individual samples in each pool cannot exceed 5, or a sensitivity of a testing assay limits a number of individual samples in each pool to be under 10. The set of the possible pooling sizes may further be determined based on a pooling technique. For example, if a pooled testing is performed based on a matrix pooling protocol, then the possible pooling sizes should be of the form M×N where M is a number of row pools and N is a number of column pools. It should be appreciated that M and N is not necessarily different or the same. When the determination of the set of the possible pooling sizes are based on both s sensitivity of a testing assay and a matrix pooling protocol, an exemplary set of the possible pooling sizes is {1×4, 4×4, 1×5, 5×5}. The set of the possible prevalence of the pathogen is determined based on the prevalence of the pathogen in each sample set. A maximum possible prevalence is less than or equal to a largest prevalence of the pathogen in all sample sets, and a minimum possible prevalence is greater than or equal to a smallest prevalence of the pathogen in all sample sets. It should be appreciated that a maximum possible prevalence may be greater than a largest prevalence of the pathogen in all sample sets in some embodiments, where a testing sample may be combined with some known positive samples. It should be also appreciated that a minimum possible prevalence may be less than a smallest prevalence of the pathogen in all sample sets in some embodiments, where a testing sample may be combined with some known negative samples, or samples from a relatively low prevalence region or population.

At block 2820, a number of initial tests to be performed for a possible pooling size in the set of the possible pooling sizes is determined. The number of the initial tests may be determined by calculating a number of pools corresponding to the possible pooling size. For example, when a possible pooling size is 4×4 in a matrix pooling test, an initial test is done per row pool and column pool. Because there are 4 rows and 4 columns in the 4×4 matrix, the initial test number is 8 (=4+4). It should be appreciated that the number of the initial tests may be determined by a method other than counting. For example, in a double pooling design, a graph may help determine the number of the initial tests.

At block 2825, a number of retests to be performed for a combination of a possible pooling size in the set of the possible pooling sizes and a possible prevalence in the set of the possible prevalence is predicted. The prediction may comprise calculating an expected number of retests based on the possible prevalence for the possible pooling size according to a pooling design and providing the expected number of the retests. For example, when a possible pooling size is 4×4 in a matrix pooling test and a possible prevalence is 5%, a binomial distribution may help predict a number of possible positive samples in the 4×4 matrix (shown in FIG. 5C). There is a 44% that no sample is positive, a 37.1% that one sample are positive, a 14.6% of two positive samples, a 3.6% of three positives, and a 0.7% of more than three positive samples. In each situation, a number of expected retests can be determined. For example, if no samples are positive, initial tests will return all negative pools, and no retests are required. If one sample is positive, initial tests probably will return one positive row pool and one positive column pool, and the intersection of the two pools correspond to a positive sample, and no retests are demanded either. If four samples are positive, the matrices in FIG. 5C illustrate different possible arrangements of the four samples and their corresponding possibilities and numbers of retests. A predicted number of retests can be determined based on the arrangements, the possibilities, and the number of retests required under different arrangements. Detailed techniques to predict a retest number is illustrated in subchapter II. A. It should be appreciated that neither matrix pooling nor binomial distribution is the only way to predict a number of retests. Other pooling techniques and mathematical methods may be adopted to perform the number prediction. In various embodiments, a number of retests is predicted based on an assumption that a retest is performed on an individual-testing basis. In some embodiments, a number of retests is predicted based on an assumption that a retest is performed on a non-individual-testing basis.

At block 2830, an optimal selection plan is determined based on the pooled testing objective function, where the optimal selection plan comprises an optimal ratio to combine samples in one or more sample sets, an optimal prevalence in a combined sample set, and an optimal pooling design for the pooled testing. The optimal selection plan provides an optimal combination of a ratio that determines how samples from different sample selection candidate sets are combined, a prevalence in a combined sample set, and a corresponding pooling design that provides a pool size and/or a pooled testing protocol. When samples are selected from sample selection candidate sets according to the optimal plan or the optimal combination, the pooled testing objective function outputs a minimum or maximum value compared with other combinations of a ratio, a prevalence, and a pooling design. For example, if a pooled testing objective function is a function to minimize a number of total tests, then an optimal selection plan provides a technique to combine samples so that a number of total tests under the optimal selection plan is the smallest comparing against numbers under other selection plan. In some embodiments, the determination of the optimal selection plan comprises determining a value of the pooled testing objective function for a combination of a possible pooling size and a prevalence; determining an optimal combination of an optimal pooling size and an optimal prevalence, where the optimal combination of the optimal pooling size and the optimal prevalence yields a greatest or a smallest value of the pooled testing objective function; determining an optimal ratio to combine samples in one or more sample sets to form a combined sample set, where a prevalence in the combined sample set equals to the optimal prevalence; determining an optimal pooling design for the pooled testing, where the optimal pooling design comprises the optimal pooling size; and providing an optimal selection plan, where the optimal selection plan comprises the optimal ratio to combine the samples in the one or more sample sets, the optimal prevalence in the combined sample set, and the optimal pooling design for the pooled testing.

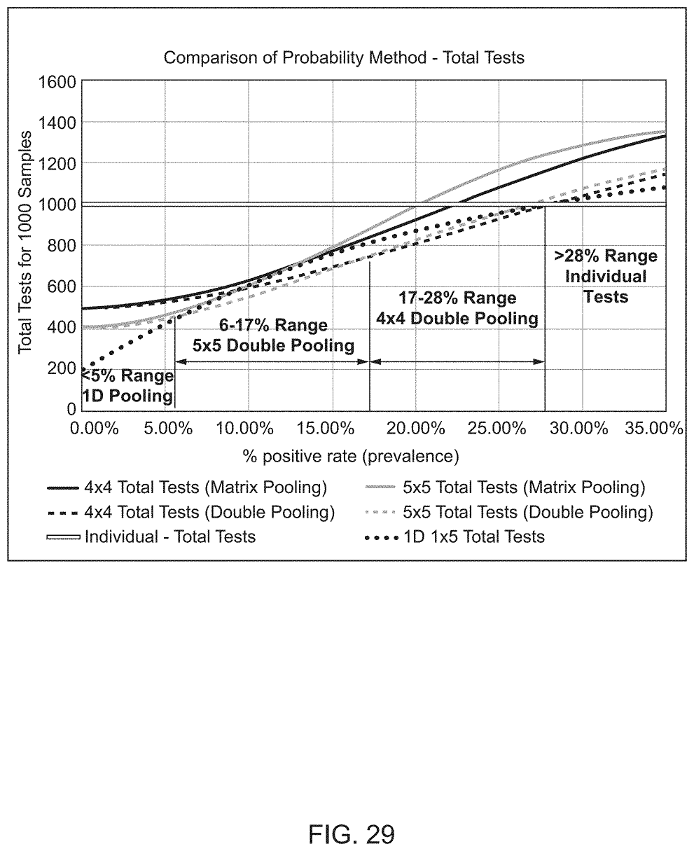

FIG. 29 illustrates one exemplary embodiment to a method using a decision graph to determine an optimal selection plan. For example, dashed curves in FIG. 29 illustrate a prediction of numbers of total tests of 1000 samples using double pooling methods, solid curves suggest predicted numbers of total tests of 1000 samples using matrix pooling methods, and the horizontal straight line illustrates that 1000 tests are needed if testing is performed individually.

The intersections suggest turning points of choosing different methods. When a pooled testing objective function is a function to minimize a number of total tests, FIG. 29 may help determine an optimal selection plan. According to FIG. 29, when a prevalence of a pathogen is lower than about 5%, 1D pooling may yield the least number of total tests (about 200-400 total test); if a prevalence is between 6%-17%, using a double pooling method to generate a 5×5 matrix may achieve the least number of total tests; for a prevalence between 17%-28%, using a double pooling method to generate a 4×4 matrix may achieve the least number of total tests; and when a prevalence is over 28%, individual testing is the best technique. Sometimes a 5×5 pooling may be unavailable based on policy reasons or sensitivity of a testing assay. In such instances, the corresponding curves may be removed from a decision graph similar to FIG. 29 to determine an optimal selection plan. Sometimes more pooling techniques or more pooling sizes may be available and similar decision graphs can be constructed like FIG. 29 to help determine an optimal selection plan. Moreover, FIG. 29 shows an instance where a pooled testing objective function is a function to minimize a number of total tests, it should be appreciated that other pooled testing objective functions may also be used to determine an optimal selection plan and similar decision graphs may be constructed using a similar way.

Although the intelligent selection machine is introduced on a step-by-step basis above, it should be appreciated that a machine learning model or the like may be implemented to perform a similar set of functions described above or in FIG. 28. For example, a machine learning model may obtain similar sample set information and pooled testing objective function as training input, and infer or predict an optimal plan as training output. Using conventional machine learning techniques such as a support vector machine, back propagation, and the like to learn from training input and output, the machine learning model will learn a set of model parameters for the machine to determine an output or an optimal selection plan for a real-time input. It is also possible that an unsupervised machine learning technique is used to provide an optimal selection plan without learning a pooled testing objective function. A neural networking technique may also be used to substitute a step-by-step intelligent selection machine to provide an optimal selection plan. It should be appreciated that a machine learning model or a neural network may substitute all or a part of the process illustrated in FIG. 28.

### III. EXAMPLES

The systems and methods implemented in various embodiments may be better understood by referring to the following examples.

### Example 1

The following example documents that the performance characteristics of the qualitative RT-PCR test are reliable and suitable for the detection of RNA from the COVID-19 virus in upper and lower respiratory specimens (such as nasopharyngeal or oropharyngeal swabs, sputum, lower respiratory tract aspirates, bronchoalveolar lavage, and nasopharyngeal wash/aspirate or nasal aspirate) when samples are combined together in pools of N=4 or N=5.

Samples were prepared according to the lab's SARS-CoV-2 Detection by Nucleic Acid Amplification (LabCorp EUA—384 Well Multiplex) standard operating procedure. However, prior to sample extraction, 50 uL of each sample for pools of 4 or 40 uL of each sample for pools of 5 were combined for testing

### Limit of Detection Validation

Validation Method

To determine the limit of detection when samples are pooled, a well characterized positive sample (2e5 cp/uL) was diluted into negative sample matrix (Saline—0.9% NaCl) to concentrations of 500, 250, 125, 62.5, and 31.25 cp/RXN (copies per reaction). 50 uL of each dilution was combined with 20 pools of negative matrix sample for the N=4 pools and 40 uL was combined with 20 pools of negative matrix for the N=5 pools. Negative pools were created by combining 145 negative samples together individually into pools of 4 or 5. Pooled samples were then processed using the LabCorp COVID-19 RT-PCR Test. Expressed per unit of volume, for unpooled samples, the LOD was 3.125 cp/μL for unpooled and 12.5 cp/μL pooled.

Validation Result

The results of the limit of detection validation produced a limit of detection of 62.5 cp/RXN for both pools of N=4 and N=5 (Table 1). The Limit of Detection of the LabCorp COVID-19 RT-PCR Test on individual samples is 15.625 cp/RXN representing a 4× loss of sensitivity when samples are combined for testing in pools of N=4 or N=5.

TABLE 1
Results of LOD Validation

| Copies | 500 | 250 | 125 | 62.5 | 30.125 |
|---|---|---|---|---|---|
| N = 4 | 20/20 | 20/20 | 20/20 | 20/20 | 18/20 |
| N = 5 | 20/20 | 20/20 | 20/20 | 19/20 | 13/20 |

### Clinical Concordance Evaluation

Validation Method

To assess sample pooling with clinical samples, randomly chosen known positives were combined with either 3 or 4 negative samples to create sample pools of N=4 or N=5. The negative sample matrix was created by individually pooling 145 negative clinical samples into 49 pools of N=4 or N=3 before combination with either a single positive or an additional negative to create the final testing pools of N=4 and N=5.39 positive samples were used for pooling along with 20 negative samples.

Once the average cycle threshold (Ct) difference between pooled and individual samples was determined, an analysis of a large database of regionally diverse clinical positive samples within the labs COVID-19 RT-PCR testing dataset was assayed to calculate what percentage of positive samples might be missed due to sample Ct dilution by combining samples into pools of N=4 or N=5.

Finally, to rule out any assay bias as a result of pooling samples, a Passing-Bablock regression analysis was performed. The “mcreg” function in the R package “mcr” was used to perform the analysis. mcreg is used to compare two measurement methods by means of regression analysis. We chose the method, “PaBa”—Passing-Bablok regression for this analysis.

Validation Result

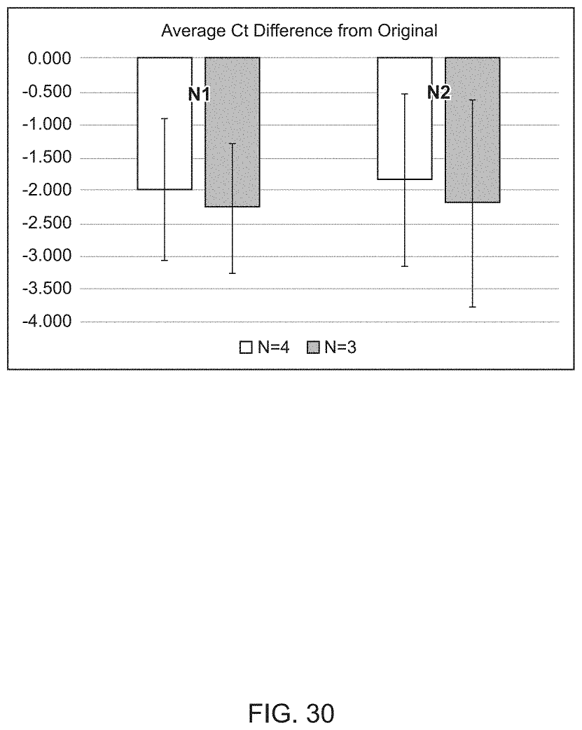

For both N=4 (Table 2) and N=5 (Table 3) pools, 38/39 positive pools were positive and 20/20 negative pools were negative. The average difference between the original Ct and the pooled Ct for N=4 was −1.972 for N1 and −1.855 for N2, and for N=5 the average difference was −2.268 for N1 and −2.208 for N2 (See FIG. 30) confirming a slight loss of assay sensitivity due to sample dilution within the pools. For the samples that resulted as indeterminate by pooling, each was positive for N1 but Undetermined for N2. All samples in these pools would be repeated as individuals according to the decision matrix in the LabCorp Sample Pooling SOP.

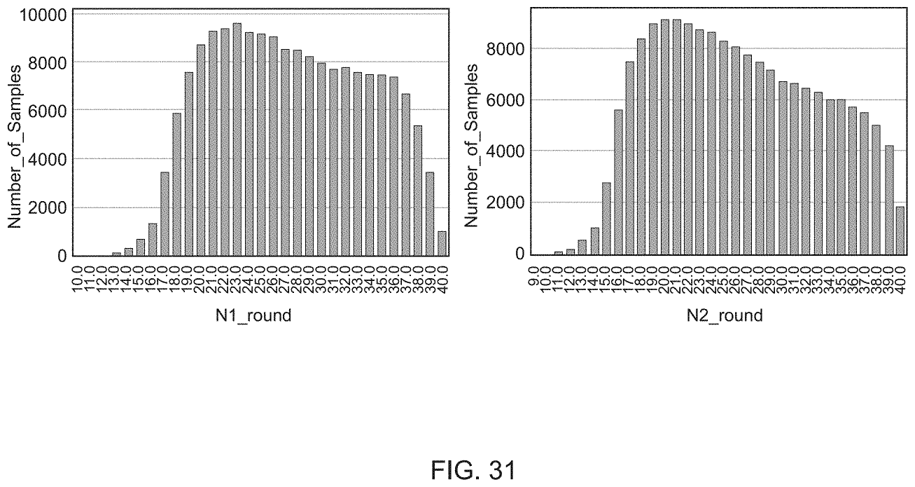

To determine how many positive samples might be missed due to sample pooling dilution, the average N1 and N2 Ct difference (2) from the clinical sample evaluation was added to 178,952 positive sample results (FIG. 31) and the number of samples that then had both N1 and N2 Ct>40 was calculated. 4,175 samples resulted in a Ct>40 after this addition, indicating that 2.3% of samples within the dataset would be missed using an N=4 or N=5 pooling strategy.

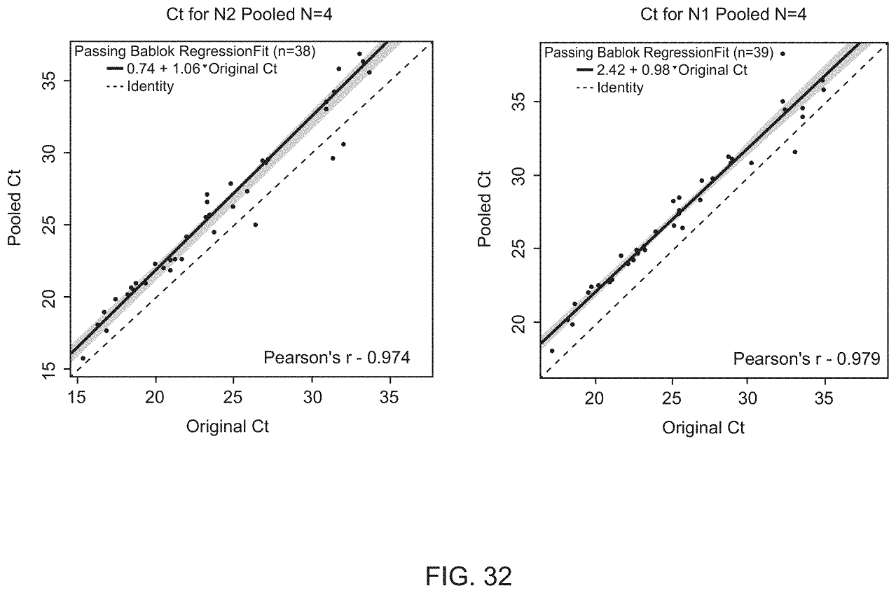

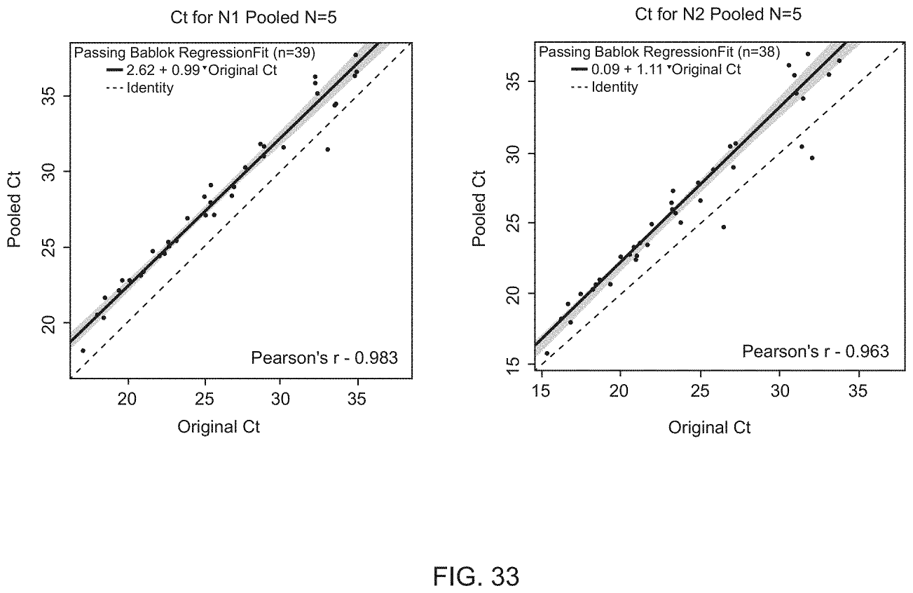

To rule out any assay bias due to pooling, particularly in low viral concentration samples, a Passing-Bablock regression analysis was performed to compare individual Cts to Pooled Cts for N=4 (FIG. 32) and N=5 (FIG. 33) pools. Results of this analysis can be seen in Table 4; however, the regression slope for N1 and N2 in either N=4 or N=5 pooling strategies is approximately 1 with R2 between 0.96 and 0.98, indicating that there is a strong linear relationship and no bias introduced by pooling samples for testing.

TABLE 2
N = 4 Sample Pooling Results

| | | Clinical Pools N = 4 | | | | | |
|---|---|---|---|---|---|---|---|
| | | Pooled Ct | | | Original Ct | | |
| | Sample | N1 | N2 | RP | N1 | N2 | RP |
| Negative | 191NP450010 | Undetermined | Undetermined | 26.106 | N/A | N/A | N/A |
| | 191NP450020 | Undetermined | Undetermined | 26.727 | N/A | N/A | N/A |
| | 191NP450030 | Undetermined | Undetermined | 25.257 | N/A | N/A | N/A |
| | 191NP450040 | Undetermined | Undetermined | 24.81 | N/A | N/A | N/A |
| | 191NP450050 | Undetermined | Undetermined | 24.621 | N/A | N/A | N/A |
| | 191NP450060 | Undetermined | Undetermined | 23.119 | N/A | N/A | N/A |
| | 191NP450070 | Undetermined | Undetermined | 28.145 | N/A | N/A | N/A |
| | 191NP450080 | Undetermined | Undetermined | 28.685 | N/A | N/A | N/A |
| | 191NP450090 | Undetermined | Undetermined | 27.574 | N/A | N/A | N/A |
| | 191NP450100 | Undetermined | Undetermined | 28.858 | N/A | N/A | N/A |
| | 191NP450110 | Undetermined | Undetermined | 25.07 | N/A | N/A | N/A |
| | 191NP450120 | Undetermined | Undetermined | 27.387 | N/A | N/A | N/A |
| | 191NP450130 | Undetermined | Undetermined | 27.293 | N/A | N/A | N/A |
| | 191NP450140 | Undetermined | Undetermined | 26.214 | N/A | N/A | N/A |
| | 191NP450150 | Undetermined | Undetermined | 24.869 | N/A | N/A | N/A |
| | 191NP450160 | Undetermined | Undetermined | 28.634 | N/A | N/A | N/A |
| | 191NP450170 | Undetermined | Undetermined | 28.344 | N/A | N/A | N/A |
| | 191NP450180 | Undetermined | Undetermined | 32.633 | N/A | N/A | N/A |
| | 191NP450190 | Undetermined | Undetermined | 27.499 | N/A | N/A | N/A |
| | 191NP450200 | Undetermined | Undetermined | 26.8 | N/A | N/A | N/A |
| Positive | 191PP450010 | 31.122 | 29.544 | 27.727 | 28.883 | 27.210 | 26.931 |
| | 191PP450020 | 26.168 | 24.211 | 26.492 | 23.868 | 21.918 | 33.001 |
| | 191PP450030 | 27.566 | 25.614 | 24.915 | 25.415 | 23.257 | 26.387 |
| | 191PP450040 | 20.108 | 18.046 | 23.355 | 18.057 | 16.273 | 26.856 |
| | 191PP450050 | 22.47 | 20.667 | 26.348 | 20.100 | 18.443 | 31.121 |
| | 191PP450060 | 30.855 | 29.425 | 26.084 | 28.838 | 27.067 | 29.757 |
| | 191PP450070 | 26.351 | 24.544 | 24.614 | 25.619 | 23.726 | 29.774 |
| | 191PP450080 | 24.243 | 22.098 | 26.527 | 22.387 | 20.560 | 28.683 |
| | 191PP450090 | 28.323 | 26.337 | 25.107 | 26.795 | 24.983 | 29.339 |
| | 191PP450100 | 31.249 | 29.467 | 26.585 | 28.661 | 26.874 | 28.718 |
| | 191PP450110 | 22.019 | 19.874 | 29.555 | 19.437 | 17.494 | 28.576 |
| | 191PP450120 | 17.987 | 15.767 | 42.695 | 17.132 | 15.408 | 34.208 |
| | 191PP450130 | 26.538 | 25.016 | 27.531 | 25.076 | 26.439 | 32.849 |
| | 191PP450140 | 27.365 | 25.676 | 25.44 | 25.391 | 23.424 | 27.203 |
| | 191PP450150 | 23.959 | 21.909 | 30.139 | 22.065 | 20.943 | 28.909 |
| | 191PP450160 | 29.582 | 27.893 | 21.97 | 26.897 | 24.808 | 27.344 |
| | 191PP450170 | 25.101 | 22.706 | 25.798 | 23.109 | 21.189 | 26.814 |
| | 191PP450180 | 29.746 | 27.369 | 25.719 | 27.646 | 25.852 | 30.747 |
| | 191PP450190 | 21.204 | 18.968 | 28.853 | 18.524 | 16.661 | 36.827 |
| | 191PP450200 | 19.788 | 17.732 | 31.127 | 18.443 | 16.849 | 27.495 |
| | 191PP450210 | 22.746 | 20.98 | 29.535 | 20.893 | 19.362 | 24.909 |
| | 191PP450220 | 22.872 | 20.964 | 25.291 | 20.982 | 18.691 | 24.01 |
| | 191PP450230 | 28.402 | 27.089 | 24.42 | 25.403 | 23.225 | 30.216 |
| | 191PP450240 | 24.848 | 22.642 | 26.299 | 22.652 | 20.842 | 26.835 |
| | 191PP450250 | 24.934 | 22.649 | 24.713 | 23.186 | 21.647 | 26.575 |
| | 191PP450260 | 24.443 | 22.381 | 27.772 | 21.612 | 19.993 | 24.893 |
| | 191PP450270 | 24.699 | 22.519 | 24.916 | 22.703 | 20.972 | 31.34 |
| | 191PP450280 | 22.371 | 20.126 | 30.502 | 19.673 | 18.251 | 33.285 |
| | 191PP450290 | 28.224 | 26.529 | 24.686 | 25.016 | 23.305 | 23.957 |
| | 197PP450010 | 36.421 | 35.595 | 25.802 | 34.781 | 33.698 | 27.197 |
| | 197PP450020 | 34.504 | 33.572 | 26.798 | 32.329 | 30.874 | 28.848 |
| | 197PP450030 | 36.454 | 36.347 | 26.340 | 34.83 | 33.326 | 29.617 |
| | 197PP450040 | 38.249 | Undetermined | 24.415 | 32.186 | 30.533 | 31.174 |
| | 197PP450050 | 35.016 | 35.912 | 26.361 | 32.202 | 31.733 | 28.052 |
| | 197PP450060 | 35.859 | 36.853 | 27.935 | 34.871 | 33.061 | 31.226 |
| | 197PP450070 | 34.550 | 34.277 | 26.057 | 33.523 | 31.452 | 30.651 |
| | 197PP450080 | 31.588 | 30.646 | 25.217 | 33.015 | 31.985 | 24.4 |
| | 197PP450090 | 30.884 | 29.587 | 24.562 | 30.162 | 31.349 | 27.979 |
| | 197PP450100 | 33.968 | 33.089 | 24.949 | 33.507 | 31.002 | 30.208 |

TABLE 3
N = 5 Sample Pooling Results

| | | Clinical Pools N = 5 | | | | | |
|---|---|---|---|---|---|---|---|
| | | Pooled Ct | | | Original Ct | | |
| | Sample | N1 | N2 | RP | N1 | N2 | RP |
| Negative | 191NP550010 | Undetermined | Undetermined | 25.135 | N/A | N/A | N/A |
| | 191NP550020 | Undetermined | Undetermined | 27.413 | N/A | N/A | N/A |
| | 191NP550030 | Undetermined | Undetermined | 24.187 | N/A | N/A | N/A |
| | 191NP550040 | Undetermined | Undetermined | 25.226 | N/A | N/A | N/A |
| | 191NP550050 | Undetermined | Undetermined | 24.965 | N/A | N/A | N/A |
| | 191NP550060 | Undetermined | Undetermined | 23.013 | N/A | N/A | N/A |
| | 191NP550070 | Undetermined | Undetermined | 26.652 | N/A | N/A | N/A |
| | 191NP550080 | Undetermined | Undetermined | 28.194 | N/A | N/A | N/A |
| | 191NP550090 | Undetermined | Undetermined | 27.755 | N/A | N/A | N/A |
| | 191NP550100 | Undetermined | Undetermined | 27.674 | N/A | N/A | N/A |
| | 191NP550110 | Undetermined | Undetermined | 25.457 | N/A | N/A | N/A |
| | 191NP550120 | Undetermined | Undetermined | 27.537 | N/A | N/A | N/A |
| | 191NP550130 | Undetermined | Undetermined | 26.986 | N/A | N/A | N/A |
| | 191NP550140 | Undetermined | Undetermined | 26.193 | N/A | N/A | N/A |
| | 191NP550150 | Undetermined | Undetermined | 25.307 | N/A | N/A | N/A |
| | 191NP550160 | Undetermined | Undetermined | 29.707 | N/A | N/A | N/A |
| | 191NP550170 | Undetermined | Undetermined | 27.826 | N/A | N/A | N/A |
| | 191NP550180 | Undetermined | Undetermined | 30.071 | N/A | N/A | N/A |
| | 191NP550190 | Undetermined | Undetermined | 27.82 | N/A | N/A | N/A |
| | 191NP550200 | Undetermined | Undetermined | 26.261 | N/A | N/A | N/A |
| Positive | 191PP550010 | 31.572 | 30.612 | 27.713 | 28.883 | 27.210 | 26.931 |
| | 191PP550020 | 26.854 | 24.898 | 26.553 | 23.868 | 21.918 | 33.001 |
| | 191PP550030 | 27.939 | 25.953 | 24.857 | 25.415 | 23.257 | 26.387 |
| | 191PP550040 | 20.467 | 18.151 | 25.882 | 18.057 | 16.273 | 26.856 |
| | 191PP550050 | 22.799 | 20.592 | 28.527 | 20.100 | 18.443 | 31.121 |
| | 191PP550060 | 30.936 | 28.957 | 26.671 | 28.838 | 27.067 | 29.757 |
| | 191PP550070 | 27.082 | 25.024 | 24.338 | 25.619 | 23.726 | 29.774 |
| | 191PP550080 | 24.479 | 22.717 | 26.798 | 22.387 | 20.560 | 28.683 |
| | 191PP550090 | 28.347 | 26.578 | 24.42 | 26.795 | 24.983 | 29.339 |
| | 191PP550100 | 31.756 | 30.433 | 26.688 | 28.661 | 26.874 | 28.718 |
| | 191PP550110 | 22.097 | 19.972 | 29.129 | 19.437 | 17.494 | 28.576 |
| | 191PP550120 | 18.073 | 15.719 | 37.992 | 17.132 | 15.408 | 34.208 |
| | 191PP550130 | 27.037 | 24.674 | 27.502 | 25.076 | 26.439 | 32.849 |
| | 191PP550140 | 27.851 | 25.709 | 26.03 | 25.391 | 23.424 | 27.203 |
| | 191PP550150 | 24.359 | 22.376 | 23.941 | 22.065 | 20.943 | 28.909 |
| | 191PP550160 | 28.946 | 27.798 | 24.943 | 26.897 | 24.808 | 27.344 |
| | 191PP550170 | 25.382 | 23.534 | 25.068 | 23.109 | 21.189 | 26.814 |
| | 191PP550180 | 30.243 | 28.731 | 25.012 | 27.646 | 25.852 | 30.747 |
| | 191PP550190 | 21.603 | 19.265 | 27.402 | 18.524 | 16.661 | 36.827 |
| | 191PP550200 | 20.284 | 17.941 | 28.694 | 18.443 | 16.849 | 27.495 |
| | 191PP550210 | 23.083 | 20.645 | 31.372 | 20.893 | 19.362 | 24.909 |
| | 191PP550220 | 23.266 | 20.869 | 25.1 | 20.982 | 18.691 | 24.01 |
| | 191PP550230 | 29.074 | 27.22 | 24.423 | 25.403 | 23.225 | 30.216 |
| | 191PP550240 | 25.257 | 23.224 | 26.62 | 22.652 | 20.842 | 26.835 |
| | 191PP550250 | 25.368 | 23.462 | 26.194 | 23.186 | 21.647 | 26.575 |
| | 191PP550260 | 24.662 | 22.577 | 26.224 | 21.612 | 19.993 | 24.893 |
| | 191PP550270 | 24.998 | 22.63 | 26.506 | 22.703 | 20.972 | 31.34 |
| | 191PP550280 | 22.739 | 20.307 | 28.916 | 19.673 | 18.251 | 33.285 |
| | 191PP550290 | 28.306 | 25.95 | 24.945 | 25.016 | 23.305 | 23.957 |
| | 197PP550010 | 36.331 | 36.520 | 26.125 | 34.781 | 33.698 | 27.197 |
| | 197PP550020 | 35.130 | 35.485 | 26.018 | 32.329 | 30.874 | 28.848 |
| | 197PP550030 | 37.679 | Undetermined | 25.576 | 34.83 | 33.326 | 29.617 |
| | 197PP550040 | 35.824 | 36.167 | 24.523 | 32.186 | 30.533 | 31.174 |
| | 197PP550050 | 36.288 | 36.998 | 26.295 | 32.202 | 31.733 | 28.052 |
| | 197PP550060 | 36.525 | 35.500 | 28.079 | 34.871 | 33.061 | 31.226 |
| | 197PP550070 | 34.420 | 33.826 | 26.489 | 33.523 | 31.452 | 30.651 |
| | 197PP550080 | 31.398 | 29.644 | 25.570 | 33.015 | 31.985 | 24.4 |
| | 197PP550090 | 31.546 | 30.390 | 24.978 | 30.162 | 31.349 | 27.979 |
| | 197PP550100 | 34.330 | 34.211 | 24.433 | 33.507 | 31.002 | 30.208 |

Table 4: Summary of Passing-Bablock Analysis. LCI and UCI are estimates of upper and lower 95% confidence intervals for the slope and Intercept. The R2 value is a Pearson's r estimated from the model.

| | Pool | | Intercept | Intercept | | Slope | Slope | |
|---|---|---|---|---|---|---|---|---|
| Target | Size | Intercept | LCI | UCI | Slope | LCI | UCI | R2 |
| N1 | 4 | 2.423 | 0.914 | 3.779 | 0.981 | 0.928 | 1.046 | 0.979 |
| N1 | 4 | 0.745 | −0.805 | 2.130 | 1.061 | 0.999 | 1.122 | 0.974 |
| N2 | 5 | 2.620 | 1.018 | 4.273 | 0.986 | 0.921 | 1.057 | 0.983 |
| N2 | 5 | 0.095 | −2.130 | 1.619 | 1.107 | 1.027 | 1.203 | 0.963 |

### Pooling Strategy Overview

Traditionally, pooling employs a 2-stage approach where samples are tested as pools and then any positive pools are retested at a later time to determine which individual was positive. While this approach saves reagents, it is not practical to implement in a high throughput testing environment where many thousands of samples would need to be pulled and retested every day. Matrix based pooling strategies allow the lab to test samples as pools while preventing the need to retest individual samples as long as the expected (and observed) number of positive samples per matrix is less than or equal to 1 (Table 5). To combat the retest problem, a matrix pooling strategy can be where samples will be tested twice in pools of 4 samples which increases lab efficiency by a factor of 2 if the tested population prevalence remains <6% (Table 5).

TABLE 5
Matrix Based Pooling Strategies Increase Throughput Without
Requiring Retesting. Green - <1 positive per matrix at indicated
prevalence, red - >1 positive per matrix at indicated
prevalence (binomial distribution used).

| Prevalence | Expected Positives | | | |
|---|---|---|---|---|
| (%) | 3 × 3 | 4 × 4 | 5 × 5 | 10 × 10 |
| 0.1 | 0.009 | 0.016 | 0.025 | 0.1 |
| 0.5 | 0.045 | 0.08 | 0.125 | 0.5 |
| 1 | 0.09 | 0.16 | 0.25 | 1 |
| 3 | 0.27 | 0.48 | 0.75 | 3 |
| 5 | 0.45 | 0.8 | 1.25 | 5 |
| 10 | 0.9 | 1.6 | 2.5 | 10 |
| 15 | 1.35 | 2.4 | 3.75 | 15 |
| Throughput | 1.5 | 2 | 2.5 | 5 |

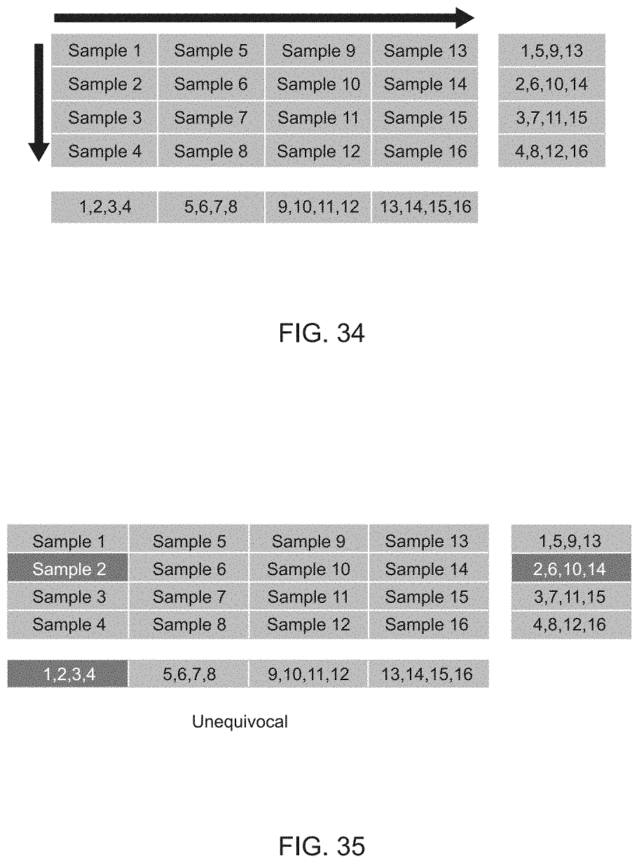

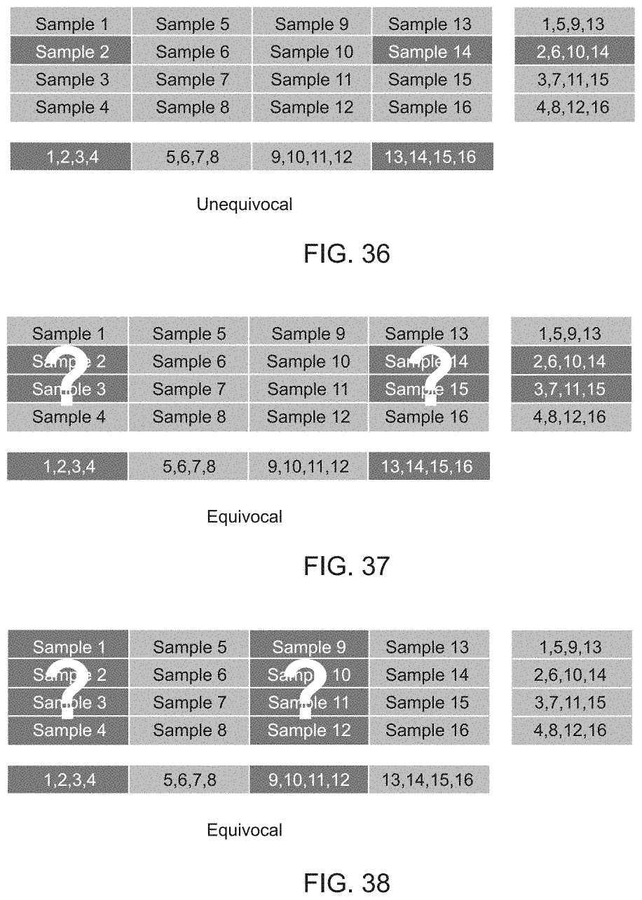

Matrix based pooling is generally straight-forward to utilize, once the appropriate matrix is determined. For example using a 4×4 matrix as an example (FIG. 34), 16 samples are arranged in a 4×4 grid. Each sample is then combined into horizontal (rows) and vertical (columns) pools to create X and Y positional information for each sample. As long as no more than 1 sample per matrix is positive, an individual positive can be ascertained without retesting any of the pools (FIG. 35). If there are 2 positives in a matrix, 40% of the time (2/5), both positives can be ascertained if they fall in either the same row or the same column (FIG. 36) while 60% of the time they will result in an equivocal result (FIG. 37). If 4 or more pools (2 per row or column set) return positive, all samples in each equivocal pool must be retested to determine which are positive (FIG. 37) as 0.4% of all four positives can be ascertained but 99.6% would be equivocal. If one or more row or column pools returns positive without a corresponding row or column pool returning positive (No X/Y intersection), then all samples within the positive pools must be retested as individuals (FIG. 38).

### Example 2

The assay can be run in a high-throughput format using 96 well plates. TECAN liquid handlers are used to transfer specimens from Saline tubes and into plates prior to sample pooling. This process is called “tube-to-plate” and results in a plate-based sample archive that feeds both the initial pooling pipeline and the retest pipeline. Sample pooling also occurs on TECAN liquid handlers dedicated to pooling. Samples are pooled in a 4×4 matrix of plates where rows and columns of samples will be stamped with the 96 head to create the final pool plates. Following testing with the LabCorp COVID-19 RT-PCR Test, pool positivity will be assessed, and positive samples ascertained based on which pools within the matrix are positive. If the results of the pooling matrix are unequivocal, e.g., more than one sample appears to be positive, all samples within both pools will be retested as individuals. At a prevalence of <5%, <1 sample per 4×4 pooling matrix will be positive. This doubles testing capacity without requiring retesting to ascertain positive samples within pools.

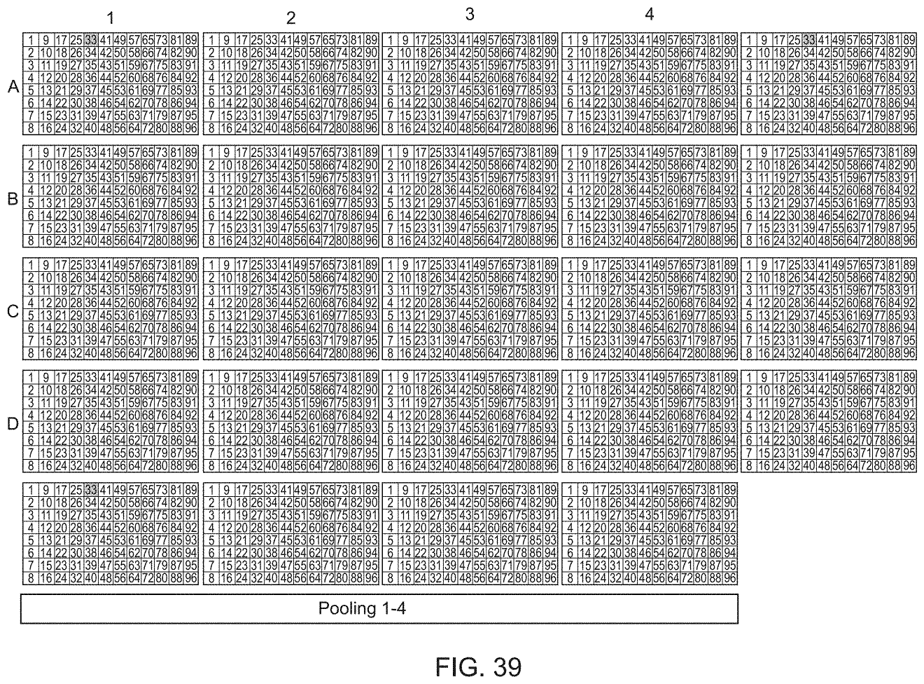

As shown in FIG. 39, pooling may be done using a 4×4 pooling process with 16 plates being combined into 8 pool plates with a 96 well pipettor. This allows the setup of 1536 samples in about 15-20 minutes. Thus, as shown in FIG. 39, 16 plates each having 96 samples, may be arranged as a 4×4 grid, and then samples from each row (horizontal) and column (vertical) pooled to give 8 pool plates. The address of the original positive sample (red or dark shading) (position 33 of the first plate) is determined by the address of the two positive pools (A-33 and 1-33).

### Example 3

The following example discloses methods for sample processing and a computer-implemented algorithm for the identification of individual positive samples.

### A. Sample Set-Up

Step 1—Archive plates are created—
Tecan pipettes 93 specimens from master tube to a single archive plate
16 archive plates are created
Each plate is labeled with a unique barcode
   For each archive plate a barcode is generated using a computerized processing system (e.g., LCWS) with a unique ID and specific requirements
For each archive plate, a plate map file produced which contains plate id, well number (well #), column (col.), row, and accession number (accession).
This plate map file is absorbed and stored. If the need arises to repeat a specimen, lookup by original specimen ID can provide a plate id, well number, row and column.
   At this point in time there is object on file that contains plate id, [row, col, well #, accession]

Step 2—Pool plates are created
Next 8 unique pool plate barcode labels are created
16 Archive plates are selected and placed on the deck of the Tecan.
   A file indicating each archive plate id, each pool plate id and deck position is sent to the computerized processing system.
The processing system absorbs pool plate file, and generates an internal map of run #, plate id, well #, row, col, [accessions]

Step 3—DNA Extraction
Each pool plate is subjected to extraction

Step 4—Hamilton Pool plates are combined into one 384 well plate (i.e. four 96 well plates)
At this point, the Hamilton Pool plate ID's and the ID of the 384 well plate are sent to the computerized processing system (LCWS).

Step 5—QS7 Results are received
The results file from the QS7 is fed into processing system (LCWS) for each of the 384 well plates

### B. Algorithm for Identification of Positive Samples

After the archive plates are created the computerized processing system will have a platemap on file based on the file received from the Tecan.

### Platemap

Plate ID
Well #
Row
Col
Accession

There are 93 rows per plate. Unique ID's will be generated for the pool plates P1-P8. The 16 archive plates will be placed on the deck of the Tecan, along with the 8 pool plates. The Tecan will scan the id of each plate and send that in a file to the processing system. Of note: position is important.

Pooling Matrix:

| | | | | |
|---|---|---|---|---|
| A1 | A2 | A3 | A4 | P1 |
| A5 | A6 | A7 | A8 | P2 |
| A9 | A10 | A11 | A12 | P3 |
| A13 | A14 | A15 | A16 | P4 |
| P5 | P6 | P7 | P8 | |

Once received, the processing system uses the combination of the Archive plate maps and the pooling file to generate a matrix internally to wait for the results from the QS7. The matrix is as follows:

### Run #

### Pool Plate Id

### Row (A-H)

### Column (1-12)

[Array of Accessions] (pulled from platemap)

### Pool Result (Neg, Pos, Delver, QC Failure)

### Status (P or C)

As the results for each pool plate are received, this matrix will be updated with the results from the individual plate (Pool result is set to the appropriate value based on the QS7 result file and Status is set to C). Once all of the statuses for the Run are set to “C”, the run can be processed into individual results for the accessions.

### Object

The processing system can then construct two internal memory structures: (a) for each set of wells an Object structure; and (b) one overall structure.
Pool Plate ID
Row
Col
Pool Result
[Array of accessions]=Result for each accession (blank to begin with)

### Array(“Pool Plate ID 1”,“Row”,“Col”)=Pool Result

Array(“Pool Plate ID 1”,“Row”,“Col”,“Accession1”)=Accession result
Array(“Pool Plate ID 1”,“Row”,“Col”,“Accession2”)=Accession result
Array(“Pool Plate ID 1”,“Row”,“Col”,“Accession3”)=Accession result
Array(“Pool Plate ID 1”,“Row”,“Col”,“Accession4”)=Accession result

### Array(“Pool Plate ID 5”,“Row”,“Col”)=Pool Result

Array(“Pool Plate ID 5”,“Row”,“Col”, “Accession1”)=Accession result
Array(“Pool Plate ID 5”,“Row”,“Col”, “Accession5”)=Accession result
Array(“Pool Plate ID 5”,“Row”,“Col”, “Accession9”)=Accession result
Array(“Pool Plate ID 5”,“Row”,“Col”, “Accession13”)=Accession result

### Overall:

Array of Accession={{Plate Id 1,Row,Col}, {Plate Id 5Row, Col} (one for row instance and one for column instance), Accession Result}.

For each instance in which the Pool result is negative, the result for each accession contained in the pool well can be marked as negative, resulted, and removed from the overall list of accessions. As each accession is marked as negative, every instance of the accession in the row/col structure on the 2^{nd} pool is marked as negative.

After all negative results have been removed and marked in the structures, a final pass can be made to determine if any remaining accessions can be resolved logically.

| P(1) | N(2) | N(3) | P(4) | P |
|---|---|---|---|---|
| N(5) | N | N | N(8) | N |
| N(9) | N | N | N(12) | N |
| N(13) | N | N | N(16) | N |
| P | N | N | P | |

In the above scenario (consider this is well A1 for the column and row) (pool plates p1, p5, p8). The structure will end up looking like this after negative have been processed.

### Array(“P1”,“1”,“A”)=POS

### Array(“P1”,“1”,“A”)=POS

### Array(“P1”,“1”,“A”,“1”)=“ ”

Array(“P1”,“1”,“A”,“2”)=Neg (because col 2 is all Neg)
Array(“P1”,“1”,“A”,“3”)=Neg (because col 3 is all Neg)

### Array(“P1”,“1”,“A”,“4”)=“ ”

### Array(“P5”,“1”,“A”)=POS

### Array(“P5”,“1”,“A”,“1”)=“ ”

### Array(“P5”,“1”,“A”,“5”)=Neg (row 2 is all Neg)

### Array(“P5”,“1”,“A”,“9”)=Neg (row 3 is all Neg)

### Array(“P5”,“1”,“A”,“13”)=Neg (row 4 is all Neg)

### Array(“P8”,“1”,“A”)=POS

### Array(“P8”,“1”,“A”,“4”)=“ ”

### Array(“P8”,“1”,“A”,“8”)=Neg (row 2 is all Neg)

### Array(“P8”,“1”,“A”,“12”)=Neg (row 3 is all Neg)

### Array(“P8”,“1”,“A”,“16”)=Neg (row 4 is all Neg)

Because the well was detected as positive (POS), yet three out of the four accessions can be ruled out based on negative results from other wells, the remaining accession can be result as detected. In this case a detected result is determined for accession 1 based on plate 5, and the detected result is determined for accession 4 based on plate 8. The detected determinations for accessions 1 and 4 carry over into plate 1, and in this scenario all 16 accessions located in well A1 can be resulted and released.

Any well within the matrix that is negative (Neg) can be used as a determination to mark the accession pooled within that well as negative. After removing all negative accessions, if a pool well is positive, and there is one and only one remaining accession in the well, then that accession can be resulted as positive. If there are more than two accessions remaining in an individual well for which a negative result cannot be determined, then the two accessions must be queued for individual testing. Indeterminate results are treated the same, only that an individual specimen that is detected as indeterminate must be queued for individual repeat testing in place of releasing a result.

### Example 4

As discussed herein, samples may be grouped based on sample origin data. Samples are sorted based on the location of the sample origin, such as, but not limited to, zip code or state. Or, samples may be sorted based upon other population demographics known to be associated with disease prevalence (e.g., specific communities, subject age, or travel history). Or, other factors associated with disease prevalence may be used. For example, in some cases samples are pre-sorted based upon zip-code. Or, samples may be sorted based on the combination of one, two three or more zip-codes, depending upon the number of samples needing testing.

Also, the sorting and/or pooling can take account for expected prevalence of the disease in a particular region. For example, samples from a region exhibiting a very low prevalence of the disease in a population (e.g., <2%) may be included in the pool group that includes samples exhibiting a relatively high prevalence of the disease in the population (>10%) such that the expected prevalence of the positive samples is optimized for the pooling procedure used (e.g., disease prevalence of about 5%). Or samples from multiple regions may be included in the pool group. For example, the pool may include about 25% of the samples from a region of high disease prevalence (e.g., >10%), 25% of the samples from a region of low disease prevalence (<1%), and about 50% of the samples from a region of average disease prevalence (about 5%) such that the pooled samples have an average disease prevalence.

Samples may be sorted at the site of procurement or in the laboratory performing the test. For example, in some cases samples are grouped at the site of procurement based on the subject's zip-code. Thus, samples from each zip-code may be pre-grouped at the procurement site for subsequent pooling at the testing lab. Or, in some cases samples are actually pooled at the site of procurement and the pooled samples sent to the testing lab. In such cases, the original samples can be maintained at the site of procurement.

### IV. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method for intelligently selecting samples to perform a pooled testing for a pathogen comprising:
obtaining samples from a plurality of regions or populations, wherein the samples from each region or population form a sample selection candidate set;
determining a prevalence of the pathogen in the samples from each region or population of the plurality of regions or populations;
determining, by an intelligent selection machine, an optimal selection plan to perform the pooled testing on the samples, wherein the optimal selection plan comprises an optimal ratio to combine the samples from the plurality of regions or populations, an optimal prevalence in a combined sample set, and an optimal pooling design for the pooled testing;
selecting samples from one or more sample selection candidate set based on the optimal ratio;
combining the selected samples to form the combined sample set with the optimal prevalence;
aliquoting the samples in the combined sample set based on the optimal pooling design;
pooling the samples in the combined sample set based on the optimal pooling design;
testing the pooled samples to determine a presence or absence of a detectable amount of the pathogen in each of the pooled samples; and
determining, based on the presence or absence of the detectable amount of the pathogen in each of the pooled samples, whether at least one individual sample comprises the detectable amount of the pathogen.

2. The method of claim 1, wherein the intelligent selection machine is configured to perform:
obtaining sample set information, wherein the sample set information comprises a size of each sample set and a prevalence of a pathogen in each sample set;
obtaining a pooled testing objective function;
determining a set of possible pooling sizes and a set of possible prevalence of the pathogen based on the sample set information;
determining a number of initial tests to be performed for a possible pooling size in the set of the possible pooling sizes;
predicting a number of retests to be performed for a combination of a possible pooling size in the set of the possible pooling sizes and a possible prevalence in the set of the possible prevalence; and
determining an optimal selection plan based on the pooled testing objective function, wherein the optimal selection plan comprises an optimal ratio to combine samples in one or more sample sets, an optimal prevalence in a combined sample set, and an optimal pooling design for the pooled testing.

3. The method of claim 2, wherein the set of the possible pooling sizes is determined based on (i) a sensitivity of a testing assay, (ii) a specification of a testing assay, (iii) the prevalence of the pathogen, (iv) a policy requirement, or (v) any combination thereof.

4. The method of claim 2, wherein the set of the possible prevalence of the pathogen is determined based on the prevalence of the pathogen in each sample set, wherein a maximum possible prevalence is less than or equal to a largest prevalence of the pathogen in all sample sets, and a minimum possible prevalence is greater than or equal to a smallest prevalence of the pathogen in all sample sets.

5. The method of claim 2, wherein the determining the optimal selection plan comprises:
determining a value of the pooled testing objective function for a combination of a possible pooling size and a prevalence;
determining an optimal combination of an optimal pooling size and an optimal prevalence, wherein the optimal combination of the optimal pooling size and the optimal prevalence yields a greatest or a smallest value of the pooled testing objective function;
determining an optimal ratio to combine samples in one or more sample sets to form a combined sample set, wherein a prevalence in the combined sample set equals to the optimal prevalence;
determining an optimal pooling design for the pooled testing, wherein the optimal pooling design comprises the optimal pooling size; and
providing an optimal selection plan, wherein the optimal selection plan comprises the optimal ratio to combine the samples in the one or more sample sets, the optimal prevalence in the combined sample set, and the optimal pooling design for the pooled testing.

6. The method of claim 1, wherein the samples comprise a specimen from either an upper or lower respiratory system.

7. The method of claim 1, wherein the pathogen is SARS-CoV-2.

8. A system comprising:
one or more data processors; and
a non-transitory computer readable storage medium containing instructions which, when executed on the one or more data processors, cause the one or more data processors to perform:
obtaining samples from a plurality of regions or populations, wherein the samples from each region or population form a sample selection candidate set;
determining a prevalence of a pathogen in the samples from each region or population of the plurality of regions or populations;
determining, by an intelligent selection machine, an optimal selection plan to perform a pooled testing on the samples, wherein the optimal selection plan comprises an optimal ratio to combine the samples from the plurality of regions or populations, an optimal prevalence in a combined sample set, and an optimal pooling design for the pooled testing;
selecting samples from one or more sample selection candidate set based on the optimal ratio;
combining the selected samples to form the combined sample set with the optimal prevalence;
aliquoting the samples in the combined sample set based on the optimal pooling design;
pooling the samples in the combined sample set based on the optimal pooling design;
testing the pooled samples to determine a presence or absence of a detectable amount of the pathogen in each of the pooled samples; and
determining, based on the presence or absence of the detectable amount of the pathogen in each of the pooled samples, whether at least one individual sample comprises the detectable amount of the pathogen.

9. The system of claim 8, wherein the intelligent selection machine is configured to perform:
obtaining sample set information, wherein the sample set information comprises a size of each sample set and a prevalence of a pathogen in each sample set;
obtaining a pooled testing objective function;
determining a set of possible pooling sizes and a set of possible prevalence of the pathogen based on the sample set information;
determining a number of initial tests to be performed for a possible pooling size in the set of the possible pooling sizes;
predicting a number of retests to be performed for a combination of a possible pooling size in the set of the possible pooling sizes and a possible prevalence in the set of the possible prevalence; and
determining an optimal selection plan based on the pooled testing objective function, wherein the optimal selection plan comprises an optimal ratio to combine samples in one or more sample sets, an optimal prevalence in a combined sample set, and an optimal pooling design for the pooled testing.

10. The system of claim 9, wherein the set of the possible pooling sizes is determined based on (i) a sensitivity of a testing assay, (ii) a specification of a testing assay, (iii) the prevalence of the pathogen, (iv) a policy requirement, or (v) any combination thereof.

11. The system of claim 9, wherein the set of the possible prevalence of the pathogen is determined based on the prevalence of the pathogen in each sample set, wherein a maximum possible prevalence is less than or equal to a largest prevalence of the pathogen in all sample sets, and a minimum possible prevalence is greater than or equal to a smallest prevalence of the pathogen in all sample sets.

12. The system of claim 9, wherein the determining the optimal selection plan comprises:
determining a value of the pooled testing objective function for a combination of a possible pooling size and a prevalence;
determining an optimal combination of an optimal pooling size and an optimal prevalence, wherein the optimal combination of the optimal pooling size and the optimal prevalence yields a greatest or a smallest value of the pooled testing objective function;
determining an optimal ratio to combine samples in one or more sample sets to form a combined sample set, wherein a prevalence in the combined sample set equals to the optimal prevalence;
determining an optimal pooling design for the pooled testing, wherein the optimal pooling design comprises the optimal pooling size; and
providing an optimal selection plan, wherein the optimal selection plan comprises the optimal ratio to combine the samples in the one or more sample sets, the optimal prevalence in the combined sample set, and the optimal pooling design for the pooled testing.

13. The system of claim 8, wherein the samples comprise a specimen from either an upper or lower respiratory system.

14. The system of claim 8, wherein the pathogen is SARS-CoV-2.

15. A computer-program product tangibly embodied in a non-transitory machine-readable storage medium, including instructions configured to cause one or more data processors to perform:
obtaining samples from a plurality of regions or populations, wherein the samples from each region or population form a sample selection candidate set;
determining a prevalence of a pathogen in the samples from each region or population of the plurality of regions or populations;
determining, by an intelligent selection machine, an optimal selection plan to perform a pooled testing on the samples, wherein the optimal selection plan comprises an optimal ratio to combine the samples from the plurality of regions or populations, an optimal prevalence in a combined sample set, and an optimal pooling design for the pooled testing;
selecting samples from one or more sample selection candidate set based on the optimal ratio;
combining the selected samples to form the combined sample set with the optimal prevalence;
aliquoting the samples in the combined sample set based on the optimal pooling design;
pooling the samples in the combined sample set based on the optimal pooling design;
testing the pooled samples to determine a presence or absence of a detectable amount of the pathogen in each of the pooled samples; and
determining, based on the presence or absence of the detectable amount of the pathogen in each of the pooled samples, whether at least one individual sample comprises the detectable amount of the pathogen.

16. The computer-program product of claim 15, wherein the intelligent selection machine is configured to perform:
obtaining sample set information, wherein the sample set information comprises a size of each sample set and a prevalence of a pathogen in each sample set;
obtaining a pooled testing objective function;
determining a set of possible pooling sizes and a set of possible prevalence of the pathogen based on the sample set information;
determining a number of initial tests to be performed for a possible pooling size in the set of the possible pooling sizes;
predicting a number of retests to be performed for a combination of a possible pooling size in the set of the possible pooling sizes and a possible prevalence in the set of the possible prevalence; and
determining an optimal selection plan based on the pooled testing objective function, wherein the optimal selection plan comprises an optimal ratio to combine samples in one or more sample sets, an optimal prevalence in a combined sample set, and an optimal pooling design for the pooled testing.

17. The computer-program product of claim 16, wherein the set of the possible pooling sizes is determined based on (i) a sensitivity of a testing assay, (ii) a specification of a testing assay, (iii) the prevalence of the pathogen, (iv) a policy requirement, or (v) any combination thereof.

18. The computer-program product of claim 16, wherein the set of the possible prevalence of the pathogen is determined based on the prevalence of the pathogen in each sample set, wherein a maximum possible prevalence is less than or equal to a largest prevalence of the pathogen in all sample sets, and a minimum possible prevalence is greater than or equal to a smallest prevalence of the pathogen in all sample sets.

19. The computer-program product of claim 16, wherein the determining the optimal selection plan comprises:
determining a value of the pooled testing objective function for a combination of a possible pooling size and a prevalence;
determining an optimal combination of an optimal pooling size and an optimal prevalence, wherein the optimal combination of the optimal pooling size and the optimal prevalence yields a greatest or a smallest value of the pooled testing objective function;
determining an optimal ratio to combine samples in one or more sample sets to form a combined sample set, wherein a prevalence in the combined sample set equals to the optimal prevalence;
determining an optimal pooling design for the pooled testing, wherein the optimal pooling design comprises the optimal pooling size; and
providing an optimal selection plan, wherein the optimal selection plan comprises the optimal ratio to combine the samples in the one or more sample sets, the optimal prevalence in the combined sample set, and the optimal pooling design for the pooled testing.

20. The computer-program product of claim 15, wherein the samples comprise a specimen from either an upper or lower respiratory system, and wherein the pathogen is SARS-CoV-2.
